(19) United States
(12) Patent Application Publication
(10) Pub. No.: US 2022/0370366 A1
(43) Pub. Date: Nov. 24, 2022

(54) TABLETS CONTAINING CILOFEXOR AND PHARMACEUTICALLY ACCEPTABLE CARRIERS

(71) Applicant: Gilead Sciences, Inc., Foster City, CA (US)

(72) Inventors: Brian Kirby, Redwood City, CA (US); Scott S. Mitchell, San Carlos, CA (US); Cara Hartz Nelson, San Mateo, CA (US); Hui-Wen Shih, San Mateo, CA (US)

(21) Appl. No.: 17/814,472

(22) Filed: Jul. 22, 2022

Related U.S. Application Data
(62) Division of application No. 16/813,314, filed on Mar. 9, 2020.
(60) Provisional application No. 62/816,771, filed on Mar. 11, 2019.

(51) Int. Cl. A61K 9/20; A61P 1/16; A61K 9/28; A61K 31/4439; A61K 31/519

(52) U.S. Cl. CPC A61K 9/2054; A61P 1/16; A61K 9/2018; A61K 9/2027; A61K 9/28; A61K 31/4439; A61K 31/519

(57) ABSTRACT

The present disclosure relates to formulations, such as tablets, of FXR agonists and therapeutic uses thereof. The disclosure also relates to methods for obtaining such formulations.

### CROSS-REFERENCE TO RELATED APPLICATIONS

This application is a divisional of U.S. Pat. application No. 16/813,314, filed Mar. 9, 2020, which application claims the benefit of priority under 35 U.S.C. § 119(e) to U.S. Provisional Patent Application No. 62/816,771, filed Mar. 11, 2019, all of which are incorporated herein by reference in their entireties.

### FIELD

The present disclosure relates to formulations, such as tablets, of FXR agonists and therapeutic uses thereof.

### BACKGROUND

The present disclosure relates to formulations of compounds that bind to the NR1H4 receptor (FXR) and act as agonists or modulators of FXR. The disclosure further relates to the uses of formulations of such compounds for the treatment and/or prophylaxis of diseases and/or conditions through binding of said nuclear receptor by said compounds.

Compounds that bind to the NR1H4 receptor (FXR) can act as agonists or modulators of FXR. FXR agonists are useful for the treatment and/or prophylaxis of diseases and conditions through binding of the NR1H4 receptor. One such FXR agonist is a compound having the following structure (hereinafter referred to as “Compound 1” or a compound of Formula (I)):

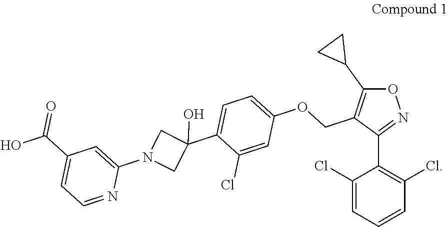 [SMILES: O=C(O)c1ccnc(N2CC(O)(c3ccc(OCc4c(-c5c(Cl)cccc5Cl)noc4C4CC4)cc3Cl)C2)c1]

Compound 1 is also known as GS-9674 or cilofexor.

### SUMMARY

The present disclosure provides, in some embodiments, a pharmaceutical composition comprising Compound 1 (also known as GS-9674 or cilofexor).

Some embodiments provided herein are directed to tablets comprising less than about 20% w/w of a Compound 1, and at least one pharmaceutically acceptable carrier, and wherein the percentage by weight is relative to the total weight of the tablet.

In some embodiments, provided herein are methods of treating a condition mediated by nonsteroidal farnesoid X receptor (FXR) in a patient in need thereof comprising administering a tablet comprising less than about 20% w/w of a Compound 1, and at least one pharmaceutically acceptable carrier, and wherein the percentage by weight is relative to the total weight of the tablet.

Some embodiments provided herein are directed to tablets comprising 3% w/w to 20% w/w of a Compound 1, and at least one pharmaceutically acceptable carrier, and wherein the percentage by weight is relative to the total weight of the tablet.

In some embodiments, provided herein are methods of treating a condition mediated by nonsteroidal farnesoid X receptor (FXR) in a patient in need thereof comprising administering a tablet comprising 3% w/w to 20% w/w of a Compound 1, and at least one pharmaceutically acceptable carrier, and wherein the percentage by weight is relative to the total weight of the tablet.

### BRIEF DESCRIPTION OF DRAWINGS

FIG. 1A and FIG. 1B depict the effect of drug load on exposure of Compound 1 as described in Example 3.

FIG. 2A and FIG. 2B depict the effect of meal type on exposure of Compound 1 as described in Example 4. In FIG. 2A, 10 mg with moderate fat meal is a 5% drug load, and all other data is with a 20% drug load.

FIG. 3A depicts the paired comparison of Compound 1 exposure from subjects who were administered Compound 1 fasted and with a high-fat meal (20% drug load). FIG. 3B illustrates the paired comparison of Compound 1 exposure from subjects who were administered Compound 1 fasted and with a high-fat meal in view of the percent change of Compound 1 exposure (20% drug load).

FIG. 4A depicts the paired comparison of Compound 1 exposure from subjects who were administered Compound 1 with a light-fat meal and with a high-fat meal (20% drug load with light fat and 12% drug load with high-fat). FIG. 4B illustrates the paired comparison of Compound 1 exposure from subjects who were administered Compound 1 with a light-fat meal and with a high-fat meal in view of the percent change of Compound 1 exposure (12% drug load).

FIG. 5 illustrates the food effect from subjects who were administered a 12% drug load of Compound 1 (within subject comparison).

FIG. 6 illustrates the percent change with light-fat meal (100 mg Compound 1, 12% drug load).

FIG. 7 illustrates the percent change with high-fat meal (100 mg Compound 1, 12% drug load).

FIG. 8 depicts the change (increase) in bioavailability when Compound 1 is administered two hours after famotidine (a representative histamine 2 receptor antagonist (H2RA)) at a 12% drug load.

FIG. 9 depicts the change (increase) in exposure (bioavailability) with famotidine pre-treatment at a 12% drug load of Compound 1.

### DETAILED DESCRIPTION

### Definitions

As used in the present disclosure, the following words and phrases are generally intended to have the meanings as set forth below, except to the extent that the context in which they are used indicates otherwise.

Unless the context requires otherwise, throughout the present specification and claims, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “pharmaceutically acceptable salt” refers to a salt prepared from pharmaceutically acceptable non-toxic bases or acids, including inorganic bases or acids and organic bases or acids. In case the compounds described herein contain one or more acidic or basic groups, the disclosure also comprises their corresponding pharmaceutically or toxicologically acceptable salts, in particular their pharmaceutically utilizable salts. Thus, the compounds described herein which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine, tris(hydroxymethyl)aminomethane (i.e. tromethamine) or amino acids. The compounds described herein which contain one or more basic groups, i.e. groups which can be protonated, can be present and can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the disclosure simultaneously contain acidic and basic groups in the molecule, the invention also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts.

A “pharmaceutical composition” refers to a formulation of a compound described herein (e.g., Compound 1) and a medium generally accepted in the art for the delivery of the biologically active compound to mammals, e.g., humans. Such a medium includes all pharmaceutically acceptable excipients therefor.

“Effective amount” or “therapeutically effective amount” refers to an amount of a compound according to the disclosure, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the disclosure which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the disclosure, and the age, body weight, general health, sex and diet of the patient. Such a therapeutically effective amount can be determined routinely by one of ordinary skill in the art having regard to their own knowledge, the state of the art, and this disclosure.

“Prevention” or “preventing” or “prophylaxis” means any treatment of a disease or condition that causes the clinical symptoms of the disease or condition not to develop. Compounds may, in some embodiments, be administered to a subject (including a human) who is at risk or has a family history of the disease or condition.

“Treating” and “treatment” of a disease include the following:

(1) preventing or reducing the risk of developing the disease, i.e., causing the clinical symptoms of the disease not to develop in a subject that may be exposed to or predisposed to the disease but does not yet experience or display symptoms of the disease,

(2) inhibiting the disease, i.e., arresting or reducing the development of the disease or its clinical symptoms, and

(3) relieving the disease, i.e., causing regression of the disease or its clinical symptoms.

The terms “subject” or “patient” refer to an animal, such as a mammal (including a human), that has been or will be the object of treatment, observation or experiment. The methods described herein may be useful in human therapy and/or veterinary applications. In some embodiments, the subject is a mammal (or the patient). In some embodiments the subject (or the patient) is human, domestic animals (e.g., dogs and cats), farm animals (e.g., cattle, horses, sheep, goats and pigs), and/or laboratory animals (e.g., mice, rats, hamsters, guinea pigs, pigs, rabbits, dogs, and monkeys). In some embodiments, the subject (or the patient) is a human.

“Human (or patient) in need thereof” refers to a human who may have or is suspected to have diseases or conditions that would benefit from certain treatment; for example, being treated with the compounds disclosed herein according to the present application.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

As used herein, the term “about” used in the context of quantitative measurements means the indicated amount ±10%. For example, “about 2:8” would mean 1.8-2.2:7.2-8.8.

Recitation of numeric ranges of values throughout the disclosure is intended to serve as a shorthand notation of referring individually to each separate value falling within the range inclusive of the values defining the range, and each separate value is incorporated in the specification as it were individually recited herein.

The term “% w/w” as used herein refers to the weight of a component based on the total weight of a composition comprising the component. For example, if component A is present in an amount of 50% w/w in a 100 mg composition, component A is present in an amount of 50 mg.

The term “carrier” or “pharmaceutically acceptable carrier” or “excipient” or “pharmaceutically acceptable excipient” refers to diluents, disintegrants, precipitation inhibitors, surfactants, glidants, binders, lubricants, and other excipients and vehicles with which the compound is administered. Carriers are generally described herein and also in “Remington's Pharmaceutical Sciences” by E. W. Martin. Examples of carriers may include, but are not limited to, aluminum monostearate, aluminum stearate, carboxymethylcellulose, carboxymethylcellulose sodium, croscarmellose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions, and the amounts of such carriers in the composition, may vary depending on the method of formulation (e.g., dry granulation formulation, solid dispersion formulation).

The term “diluent” refers to chemical compounds that are used to dilute the compound of interest prior to delivery. Diluents can also serve to stabilize compounds. Non-limiting examples of diluents include starch, saccharides, disaccharides, sucrose, lactose, lactose monohydrate, polysaccharides, cellulose, cellulose ethers, hydroxypropyl cellulose, microcrystalline cellulose, sugar alcohols, xylitol, sorbitol, maltitol, compressible sugars, calcium or sodium carbonate, dicalcium phosphate, dibasic calcium phosphate dehydrate, mannitol, and tribasic calcium phosphate.

The term “binder” when used herein relates to any pharmaceutically acceptable film which can be used to bind together the active and inert components of the carrier together to maintain cohesive and discrete portions. Non-limiting examples of binders include hydroxypropylcellulose, hydroxypropylmethylcellulose, povidone, copovidone, and ethyl cellulose.

The term “disintegrant” refers to a substance which, upon addition to a solid preparation, facilitates its break-up or disintegration after administration and permits the release of an active ingredient as efficiently as possible to allow for its rapid dissolution. Non-limiting examples of disintegrants include maize starch, sodium starch glycolate, croscarmellose sodium, crospovidone, microcrystalline cellulose, modified corn starch, sodium carboxymethyl starch, povidone, pregelatinized starch, and alginic acid.

The term “lubricant” refers to a substance added to a powder blend to prevent the compacted powder mass from sticking to the equipment during the tableting or encapsulation process. A lubricant can aid the ejection of the tablet from the dies and can improve powder flow. Non-limiting examples of lubricants include magnesium stearate, stearic acid, silica, fats, calcium stearate, polyethylene glycol, sodium stearyl fumarate, or talc; and solubilizers such as fatty acids including lauric acid, oleic acid, and C₈/C₁₀ fatty acid.

The term “film coating” refers to a thin, uniform, film on the surface of a substrate (e.g., tablet). Film coatings are particularly useful for protecting the active ingredient(s) from photolytic degradation. Non-limiting examples of film coatings include polyvinylalcohol based, hydroxyethylcellulose, hydroxypropylmethylcellulose, sodium carboxymethylcellulose, polyethylene glycol 4000 and cellulose acetate phthalate film coatings.

The term “glidant” refers to substances used in tablet and capsule formulations to improve flow-properties during tablet compression and to produce an anti-caking effect. Examples of glidants may include colloidal silicon dioxide, talc, fumed silica, starch, starch derivatives, and bentonite.

### Pharmaceutical Compositions

Provided herein are pharmaceutical compositions comprising an FXR agonist.

Some embodiments provided herein are directed to pharmaceutical compositions comprising Compound 1 or a pharmaceutically acceptable salt thereof.

Compound 1 may be synthesized and characterized using methods known to those of skill in the art, such as those described in U.S. Publication No. 2014/0221659.

In some embodiments, the pharmaceutical compositions described herein exhibit improved dissolution properties. In some embodiments, the pharmaceutical compositions described herein exhibit drug load dependent reductions in variability of drug exposure in a subject population For example, in some embodiments, the effect of reducing drug load on percent increase in exposure of Compound 1 is greater for one or more subjects that exhibit lower drug exposure from a higher drug load of Compound 1 compared to one or more subjects with higher exposure from the lower drug load of Compound 1.

In some embodiments, administering Compound 1 with a high-fat meal increases Compound 1 exposure relative to fasted conditions or administration with a light-fat or moderate fat meal.

In some embodiments, the effect of a high-fat meal on percent increase in exposure of Compound 1 is greater for subjects that exhibit lower drug exposure when Compound 1 is taken under fasted conditions or with a light-fat meal compared to subjects that exhibit higher drug exposure when Compound 1 is taken under fasted conditions or with a light-fat meal.

Some embodiments provided herein are directed to pharmaceutical compositions comprising less than about 20% w/w of a Compound 1:

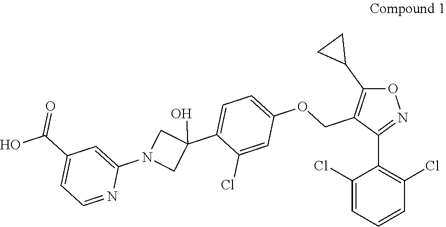 [SMILES: O=C(O)c1ccnc(N2CC(O)(c3ccc(OCc4c(-c5c(Cl)cccc5Cl)noc4C4CC4)cc3Cl)C2)c1]

and at least one pharmaceutically acceptable carrier, and wherein the percentage by weight is relative to the total weight of the pharmaceutical composition.

Some embodiments provided herein are directed to pharmaceutical compositions comprising 3% w/w to 20% w/w of a Compound 1:

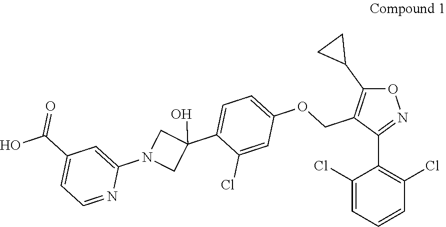 [SMILES: O=C(O)c1ccnc(N2CC(O)(c3ccc(OCc4c(-c5c(Cl)cccc5Cl)noc4C4CC4)cc3Cl)C2)c1]

and at least one pharmaceutically acceptable carrier, and wherein the percentage by weight is relative to the total weight of the pharmaceutical composition.

Some embodiments provided herein are directed to pharmaceutical compositions comprising less than about 25% w/w of a Compound 1:

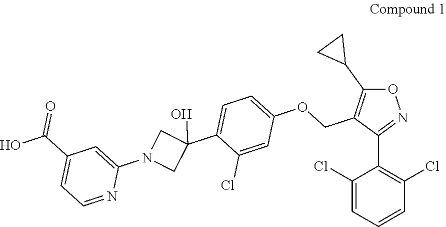 [SMILES: O=C(O)c1ccnc(N2CC(O)(c3ccc(OCc4c(-c5c(Cl)cccc5Cl)noc4C4CC4)cc3Cl)C2)c1]

or a pharmaceutically acceptable salt thereof, and at least one pharmaceutically acceptable carrier, and wherein the percentage by weight is relative to the total weight of the pharmaceutical composition.

Some embodiments provided herein are directed to pharmaceutical compositions comprising 5% w/w to 25% w/w of a Compound 1:

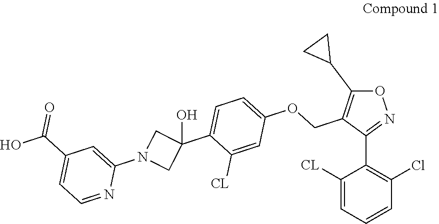

or a pharmaceutically acceptable salt thereof, and at least one pharmaceutically acceptable carrier, and wherein the percentage by weight is relative to the total weight of the pharmaceutical composition.

In certain embodiments, the pharmaceutical compositions described herein comprise a tromethamine salt of Compound 1, such as Form I, which has been shown to impart improved bioavailability relative to the zwitterion, and has suitable chemical and physical stability in the drug product.

Some embodiments provided herein are directed to pharmaceutical compositions comprising less than about 25% w/w of a tromethamine salt of Compound 1:

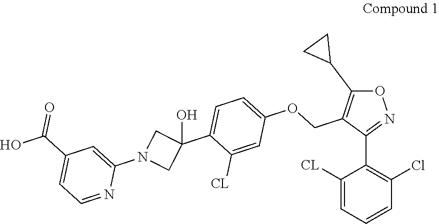

and at least one pharmaceutically acceptable carrier, and wherein the percentage by weight is relative to the total weight of the pharmaceutical composition. In some embodiments, the pharmaceutical composition comprises about 14% w/w of a tromethamine salt of Compound 1. In some embodiments, the pharmaceutical composition comprises about 6% w/w of a tromethamine salt of Compound 1. Some embodiments provided herein are directed to pharmaceutical compositions comprising 5% w/w to 20% w/w of a tromethamine salt of Compound 1:

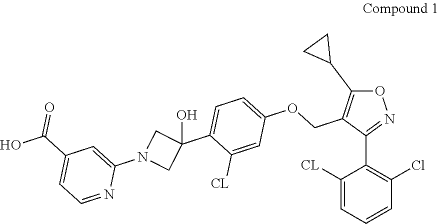

and at least one pharmaceutically acceptable carrier, and wherein the percentage by weight is relative to the total weight of the pharmaceutical composition. In some embodiments, the pharmaceutical composition comprises 14% w/w of a tromethamine salt of Compound 1. In some embodiments, the pharmaceutical composition comprises 6% w/w of a tromethamine salt of Compound 1.

In some embodiments, the pharmaceutical composition comprises about 1% w/w to about 25% w/w of Compound 1 or a pharmaceutically acceptable salt thereof. In some embodiments, the pharmaceutical composition comprises about 3% w/w to about 25% w/w of Compound 1 or a pharmaceutically acceptable salt thereof. In some embodiments, the pharmaceutical composition comprises about 5% w/w to about 25% w/w of Compound 1 or a pharmaceutically acceptable salt thereof. In some embodiments, the pharmaceutical composition comprises about 5% w/w to about 20% w/w of Compound 1 or a pharmaceutically acceptable salt thereof. In some embodiments, the pharmaceutical composition comprises about 5% w/w to about 15% w/w of Compound 1 or a pharmaceutically acceptable salt thereof. In some embodiments, the pharmaceutical composition comprises about 5% w/w to about 12% w/w of Compound 1 or a pharmaceutically acceptable salt thereof. In some embodiments, the pharmaceutical composition comprises about 5% w/w to about 10% w/w of Compound 1 or a pharmaceutically acceptable salt thereof. In some embodiments, the pharmaceutical composition comprises about 5% w/w to about 8% w/w of Compound 1 or a pharmaceutically acceptable salt thereof. In some embodiments, the pharmaceutical composition comprises about 8% w/w to about 12% w/w of Compound 1 or a pharmaceutically acceptable salt thereof.

In some embodiments, the pharmaceutical composition comprises 1% w/w to 25% w/w of Compound 1 or a pharmaceutically acceptable salt thereof. In some embodiments, the pharmaceutical composition comprises 3% w/w to 25% w/w of Compound 1 or a pharmaceutically acceptable salt thereof. In some embodiments, the pharmaceutical composition comprises 5% w/w to 25% w/w of Compound 1 or a pharmaceutically acceptable salt thereof. In some embodiments, the pharmaceutical composition comprises 5% w/w to 20% w/w of Compound 1 or a pharmaceutically acceptable salt thereof. In some embodiments, the pharmaceutical composition comprises 5% w/w to 15% w/w of Compound 1 or a pharmaceutically acceptable salt thereof. In some embodiments, the pharmaceutical composition comprises 5% w/w to 12% w/w of Compound 1 or a pharmaceutically acceptable salt thereof. In some embodiments, the pharmaceutical composition comprises 5% w/w to 10% w/w of Compound 1 or a pharmaceutically acceptable salt thereof. In some embodiments, the pharmaceutical composition comprises 5% w/w to 8% w/w of Compound 1 or a pharmaceutically acceptable salt thereof. In some embodiments, the pharmaceutical composition comprises 8% w/w to 12% w/w of Compound 1 or a pharmaceutically acceptable salt thereof.

In some embodiments, the pharmaceutical composition comprises about 3% w/w to about 25% w/w of a pharmaceutically acceptable salt of Compound 1. In some embodiments, the pharmaceutical composition comprises about 5% w/w to about 25% w/w of a pharmaceutically acceptable salt of Compound 1.

In some embodiments, the pharmaceutical composition comprises 3% w/w to 25% w/w of a pharmaceutically acceptable salt of Compound 1. In some embodiments, the pharmaceutical composition comprises 5% w/w to 25% w/w of a pharmaceutically acceptable salt of Compound 1.

In some embodiments, the pharmaceutical composition comprises less than about 30% w/w of a pharmaceutically acceptable salt of Compound 1. In some embodiments, the pharmaceutical composition comprises less than about 25% w/w of a pharmaceutically acceptable salt of Compound 1. In some embodiments, the pharmaceutical composition comprises less than about 20% w/w of a pharmaceutically acceptable salt of Compound 1. In some embodiments, the pharmaceutical composition comprises less than about 18% w/w of a pharmaceutically acceptable salt of Compound 1. In some embodiments, the pharmaceutical composition comprises less than about 15% w/w of a pharmaceutically acceptable salt of Compound 1. In some embodiments, the pharmaceutical composition comprises less than about 10% w/w of a pharmaceutically acceptable salt of Compound 1. In some embodiments, the pharmaceutical composition comprises less than about 8% w/w of a pharmaceutically acceptable salt of Compound 1. In some embodiments, the pharmaceutical composition comprises less than about 7% w/w of a pharmaceutically acceptable salt of Compound 1. In some embodiments, the pharmaceutical composition comprises less than about 6% w/w of a pharmaceutically acceptable salt of Compound 1. In some embodiments, the pharmaceutical composition comprises less than about 5% w/w of a pharmaceutically acceptable salt of Compound 1. In some embodiments, the pharmaceutical composition comprises less than about 3% w/w of a pharmaceutically acceptable salt of Compound 1.

In some embodiments, the pharmaceutical composition comprises 1% w/w to 30% w/w of a pharmaceutically acceptable salt of Compound 1. In some embodiments, the pharmaceutical composition comprises 1% w/w to 25% w/w of a pharmaceutically acceptable salt of Compound 1. In some embodiments, the pharmaceutical composition comprises 1% w/w to 20% w/w of a pharmaceutically acceptable salt of Compound 1. In some embodiments, the pharmaceutical composition comprises 1% w/w to 18% w/w of a pharmaceutically acceptable salt of Compound 1. In some embodiments, the pharmaceutical composition comprises 1% w/w to 15% w/w of a pharmaceutically acceptable salt of Compound 1. In some embodiments, the pharmaceutical composition comprises 1% w/w to 10% w/w of a pharmaceutically acceptable salt of Compound 1. In some embodiments, the pharmaceutical composition comprises 1% w/w to 8% w/w of a pharmaceutically acceptable salt of Compound 1. In some embodiments, the pharmaceutical composition comprises 1% w/w to 7% w/w of a pharmaceutically acceptable salt of Compound 1. In some embodiments, the pharmaceutical composition comprises 1% w/w to 6% w/w of a pharmaceutically acceptable salt of Compound 1. In some embodiments, the pharmaceutical composition comprises 1% w/w to 5% w/w of a pharmaceutically acceptable salt of Compound 1. In some embodiments, the pharmaceutical composition comprises 1% w/w to 3% w/w of a pharmaceutically acceptable salt of Compound 1.

In some embodiments, the pharmaceutical composition comprises about 30% w/w of a pharmaceutically acceptable salt of Compound 1. In some embodiments, the pharmaceutical composition comprises about 25% w/w of a pharmaceutically acceptable salt of Compound 1. In some embodiments, the pharmaceutical composition comprises about 20% w/w of a pharmaceutically acceptable salt of Compound 1. In some embodiments, the pharmaceutical composition comprises about 18% w/w of a pharmaceutically acceptable salt of Compound 1. In some embodiments, the pharmaceutical composition comprises about 15% w/w of a pharmaceutically acceptable salt of Compound 1. In some embodiments, the pharmaceutical composition comprises about 14% w/w of a pharmaceutically acceptable salt of Compound 1. In some embodiments, the pharmaceutical composition comprises about 12% w/w of a pharmaceutically acceptable salt of Compound 1. In some embodiments, the pharmaceutical composition comprises about 10% w/w of a pharmaceutically acceptable salt of Compound 1. In some embodiments, the pharmaceutical composition comprises about 8% w/w of a pharmaceutically acceptable salt of Compound 1. In some embodiments, the pharmaceutical composition comprises about 7% w/w of a pharmaceutically acceptable salt of Compound 1. In some embodiments, the pharmaceutical composition comprises about 6% w/w of a pharmaceutically acceptable salt of Compound 1. In some embodiments, the pharmaceutical composition comprises about 5% w/w of a pharmaceutically acceptable salt of Compound 1. In some embodiments, the pharmaceutical composition comprises about 1% w/w of a pharmaceutically acceptable salt of Compound 1.

In some embodiments, the pharmaceutical composition comprises 20% w/w of a pharmaceutically acceptable salt of Compound 1. In some embodiments, the pharmaceutical composition comprises 18% w/w of a pharmaceutically acceptable salt of Compound 1. In some embodiments, the pharmaceutical composition comprises 15% w/w of a pharmaceutically acceptable salt of Compound 1. In some embodiments, the pharmaceutical composition comprises 14% w/w of a pharmaceutically acceptable salt of Compound 1. In some embodiments, the pharmaceutical composition comprises 12% w/w of a pharmaceutically acceptable salt of Compound 1. In some embodiments, the pharmaceutical composition comprises 10% w/w of a pharmaceutically acceptable salt of Compound 1. In some embodiments, the pharmaceutical composition comprises 8% w/w of a pharmaceutically acceptable salt of Compound 1. In some embodiments, the pharmaceutical composition comprises 7% w/w of a pharmaceutically acceptable salt of Compound 1. In some embodiments, the pharmaceutical composition comprises 6% w/w of a pharmaceutically acceptable salt of Compound 1. In some embodiments, the pharmaceutical composition comprises 5% w/w of a pharmaceutically acceptable salt of Compound 1. In some embodiments, the pharmaceutical composition comprises 1% w/w of a pharmaceutically acceptable salt of Compound 1.

In some embodiments, the pharmaceutical composition comprises about 3% w/w to about 25% w/w of Compound 1 or a tromethamine salt thereof. In some embodiments, the pharmaceutical composition comprises about 5% w/w to about 25% w/w of Compound 1 or a tromethamine thereof. In some embodiments, the pharmaceutical composition comprises about 5% w/w to about 20% w/w of Compound 1 or a tromethamine salt thereof. In some embodiments, the pharmaceutical composition comprises about 5% w/w to about 15% w/w of Compound 1 or a tromethamine salt thereof. In some embodiments, the pharmaceutical composition comprises about 5% w/w to about 12% w/w of Compound 1 or a tromethamine salt thereof. In some embodiments, the pharmaceutical composition comprises about 5% w/w to about 10% w/w of Compound 1 or a tromethamine salt thereof. In some embodiments, the pharmaceutical composition comprises about 5% w/w to about 8% w/w of Compound 1 or a tromethamine thereof. In some embodiments, the pharmaceutical composition comprises about 8% w/w to about 12% w/w of Compound 1 or a tromethamine salt thereof.

In some embodiments, the pharmaceutical composition comprises 3% w/w to 25% w/w of Compound 1 or a tromethamine salt thereof. In some embodiments, the pharmaceutical composition comprises 5% w/w to 25% w/w of Compound 1 or a tromethamine thereof. In some embodiments, the pharmaceutical composition comprises 5% w/w to 20% w/w of Compound 1 or a tromethamine salt thereof. In some embodiments, the pharmaceutical composition comprises 5% w/w to 15% w/w of Compound 1 or a tromethamine salt thereof. In some embodiments, the pharmaceutical composition comprises 5% w/w to 12% w/w of Compound 1 or a tromethamine salt thereof. In some embodiments, the pharmaceutical composition comprises 5% w/w to 10% w/w of Compound 1 or a tromethamine salt thereof. In some embodiments, the pharmaceutical composition comprises 5% w/w to 8% w/w of Compound 1 or a tromethamine thereof. In some embodiments, the pharmaceutical composition comprises 8% w/w to 12% w/w of Compound 1 or a tromethamine salt thereof.

In some embodiments, the pharmaceutical composition comprises about 3% to about 25% w/w of a tromethamine salt of Compound 1. In some embodiments, the pharmaceutical composition comprises about 5% to about 25% w/w of a tromethamine salt of Compound 1.

In some embodiments, the pharmaceutical composition comprises 3% to 25% w/w of a tromethamine salt of Compound 1. In some embodiments, the pharmaceutical composition comprises 5% to 25% w/w of a tromethamine salt of Compound 1.

In some embodiments, the pharmaceutical composition comprises less than about 25% w/w of a tromethamine salt of Compound 1. In some embodiments, the pharmaceutical composition comprises less than about 20% w/w of a tromethamine salt of Compound 1. In some embodiments, the pharmaceutical composition comprises less than about 18% w/w of a tromethamine salt of Compound 1. In some embodiments, the pharmaceutical composition comprises less than about 15% w/w of a tromethamine salt of Compound 1. In some embodiments, the pharmaceutical composition comprises less than about 10% w/w of a tromethamine of Compound 1. In some embodiments, the pharmaceutical composition comprises less than about 8% w/w of a tromethamine salt of Compound 1. In some embodiments, the pharmaceutical composition comprises less than about 7% w/w of a tromethamine salt of Compound 1. In some embodiments, the pharmaceutical composition comprises less than about 6% w/w of a tromethamine salt of Compound 1. In some embodiments, the pharmaceutical composition comprises less than about 5% w/w of a tromethamine salt of Compound 1.

In some embodiments, the pharmaceutical composition comprises 1% to 25% w/w of a tromethamine salt of Compound 1. In some embodiments, the pharmaceutical composition comprises 1% to 20% w/w of a tromethamine salt of Compound 1. In some embodiments, the pharmaceutical composition comprises 1% to 18% w/w of a tromethamine salt of Compound 1. In some embodiments, the pharmaceutical composition comprises 1% to 15% w/w of a tromethamine salt of Compound 1. In some embodiments, the pharmaceutical composition comprises 1% to 10% w/w of a tromethamine of Compound 1. In some embodiments, the pharmaceutical composition comprises 1% to 8% w/w of a tromethamine salt of Compound 1. In some embodiments, the pharmaceutical composition comprises 1% to 7% w/w of a tromethamine salt of Compound 1. In some embodiments, the pharmaceutical composition comprises 1% to 6% w/w of a tromethamine salt of Compound 1. In some embodiments, the pharmaceutical composition comprises 1% to 5% w/w of a tromethamine salt of Compound 1.

In some embodiments, the pharmaceutical composition comprises about 3% w/w to about 25% w/w of Compound 1. In some embodiments, the pharmaceutical composition comprises about 5% w/w to about 25% w/w of Compound 1. In some embodiments, the pharmaceutical composition comprises about 5% w/w to about 20% w/w of Compound 1. In some embodiments, the pharmaceutical composition comprises about 5% w/w to about 15% w/w of Compound 1. In some embodiments, the pharmaceutical composition comprises about 5% w/w to about 12% w/w of Compound 1. In some embodiments, the pharmaceutical composition comprises about 5% w/w to about 10% w/w of Compound 1. In some embodiments, the pharmaceutical composition comprises about 5% w/w to about 8% w/w of Compound 1.

In some embodiments, the pharmaceutical composition comprises 3% w/w to 20% w/w of Compound 1. In some embodiments, the pharmaceutical composition comprises 5% w/w to 20% w/w of Compound 1. In some embodiments, the pharmaceutical composition comprises 5% w/w to 15% w/w of Compound 1. In some embodiments, the pharmaceutical composition comprises 5% w/w to 12% w/w of Compound 1. In some embodiments, the pharmaceutical composition comprises 5% w/w to 10% w/w of Compound 1. In some embodiments, the pharmaceutical composition comprises 5% w/w to 8% w/w of Compound 1.

In some embodiments, the pharmaceutical composition comprises less than about 25% w/w of Compound 1. In some embodiments, the pharmaceutical composition comprises less than about 20% w/w of Compound 1. In some embodiments, the pharmaceutical composition comprises less than about 18% w/w of Compound 1. In some embodiments, the pharmaceutical composition comprises less than about 15% w/w of Compound 1. In some embodiments, the pharmaceutical composition comprises less than about 12% w/w of Compound 1. In some embodiments, the pharmaceutical composition comprises less than about 10% w/w of Compound 1. In some embodiments, the pharmaceutical composition comprises less than about 8% w/w of Compound 1. In some embodiments, the pharmaceutical composition comprises less than about 5% w/w of Compound 1.

In some embodiments, the pharmaceutical composition comprises 1% to 25% w/w of Compound 1. In some embodiments, the pharmaceutical composition comprises less than 20% w/w of Compound 1. In some embodiments, the pharmaceutical composition comprises 1% to 18% w/w of Compound 1. In some embodiments, the pharmaceutical composition comprises 1% to 15% w/w of Compound 1. In some embodiments, the pharmaceutical composition comprises 1% to 12% w/w of Compound 1. In some embodiments, the pharmaceutical composition comprises 1% to 10% w/w of Compound 1. In some embodiments, the pharmaceutical composition comprises 1% to 8% w/w of Compound 1. In some embodiments, the pharmaceutical composition comprises 1% to 5% w/w of Compound 1.

In some embodiments, the pharmaceutical composition comprises about 20% w/w of Compound 1. About 20% w/w of Compound 1 also refers to about 24% w/w of a tromethamine salt of Compound 1.

In some embodiments, the pharmaceutical composition comprises 20% w/w of Compound 1. 20% w/w of Compound 1 also refers to 24% w/w of a tromethamine salt of Compound 1.

In some embodiments, the pharmaceutical composition comprises about 18% w/w of Compound 1. In some embodiments, the pharmaceutical composition comprises about 15% w/w of Compound 1. In some embodiments, the pharmaceutical composition comprises about 12% w/w of Compound 1. In some embodiments, the pharmaceutical composition comprises about 10% w/w of Compound 1. In some embodiments, the pharmaceutical composition about 8% w/w of Compound 1. In some embodiments, the pharmaceutical composition comprises about 5% w/w of Compound 1. In some embodiments, the pharmaceutical composition comprises about 2.5% w/w of Compound 1. In some embodiments, the pharmaceutical composition comprises about 1% w/w of Compound 1.

In some embodiments, the pharmaceutical composition comprises 18% w/w of Compound 1. In some embodiments, the pharmaceutical composition comprises 15% w/w of Compound 1. In some embodiments, the pharmaceutical composition comprises 12% w/w of Compound 1. In some embodiments, the pharmaceutical composition comprises 10% w/w of Compound 1. In some embodiments, the pharmaceutical composition 8% w/w of Compound 1. In some embodiments, the pharmaceutical composition comprises 5% w/w of Compound 1. In some embodiments, the pharmaceutical composition comprises 2.5% w/w of Compound 1. In some embodiments, the pharmaceutical composition comprises 1% w/w of Compound 1.

In some embodiments, the pharmaceutical composition comprises about 200 mg to about 1 mg of Compound 1. In some embodiments, the pharmaceutical composition comprises about 150 mg to about 10 mg of Compound 1. In some embodiments, the pharmaceutical composition comprises about 125 mg to about 15 mg of Compound 1. In some embodiments, the pharmaceutical composition comprises about 100 mg to about 30 mg of Compound 1. In some embodiments, the pharmaceutical composition comprises about 100 mg to about 20 mg of Compound 1. In some embodiments, the pharmaceutical composition comprises about 50 mg to about 200 mg of Compound 1. In some embodiments, the pharmaceutical composition comprises about 50 mg to about 150 mg of Compound 1. In some embodiments, the pharmaceutical composition comprises about 10 mg to about 50 mg of Compound 1.

In some embodiments, the pharmaceutical composition comprises 200 mg to 1 mg of Compound 1. In some embodiments, the pharmaceutical composition comprises 150 mg to 10 mg of Compound 1. In some embodiments, the pharmaceutical composition comprises 125 mg to 15 mg of Compound 1. In some embodiments, the pharmaceutical composition comprises 100 mg to 30 mg of Compound 1. In some embodiments, the pharmaceutical composition comprises 100 mg to 20 mg of Compound 1. In some embodiments, the pharmaceutical composition comprises 50 mg to 200 mg of Compound 1. In some embodiments, the pharmaceutical composition comprises 50 mg to 150 mg of Compound 1. In some embodiments, the pharmaceutical composition comprises 10 mg to 50 mg of Compound 1.

In some embodiments, the pharmaceutical composition comprises about 150 mg of Compound 1. In some embodiments, the pharmaceutical composition comprises about 100 mg of Compound 1. In some embodiments, the pharmaceutical composition comprises about 90 mg of Compound 1. In some embodiments, the pharmaceutical composition comprises about 80 mg of Compound 1. In some embodiments, the pharmaceutical composition comprises about 70 mg of Compound 1. In some embodiments, the pharmaceutical composition comprises about 60 mg of Compound 1. In some embodiments, the pharmaceutical composition comprises about 50 mg of Compound 1. In some embodiments, the pharmaceutical composition comprises about 40 mg of Compound 1. In some embodiments, the pharmaceutical composition comprises about 30 mg of Compound 1. In some embodiments, the pharmaceutical composition comprises about 20 mg of Compound 1. In some embodiments, the pharmaceutical composition comprises about 10 mg of Compound 1.

In some embodiments, the pharmaceutical composition comprises 150 mg of Compound 1. In some embodiments, the pharmaceutical composition comprises 100 mg of Compound 1. In some embodiments, the pharmaceutical composition comprises 90 mg of Compound 1. In some embodiments, the pharmaceutical composition comprises 80 mg of Compound 1. In some embodiments, the pharmaceutical composition comprises 70 mg of Compound 1. In some embodiments, the pharmaceutical composition comprises 60 mg of Compound 1. In some embodiments, the pharmaceutical composition comprises 50 mg of Compound 1. In some embodiments, the pharmaceutical composition comprises 40 mg of Compound 1. In some embodiments, the pharmaceutical composition comprises 30 mg of Compound 1. In some embodiments, the pharmaceutical composition comprises 20 mg of Compound 1. In some embodiments, the pharmaceutical composition comprises 10 mg of Compound 1.

In some embodiments, the pharmaceutical composition comprises 100 mg of Compound 1, wherein Compound 1 is present in an amount of about 5% to about 12% w/w, or from about 8% to about 12% w/w. In some embodiments, the pharmaceutical composition comprises 30 mg of Compound 1, wherein Compound 1 is present in an amount of about 5% to about 12% w/w, or for example about 8% to about 12% w/w.

In some embodiments, the pharmaceutical composition comprises 100 mg of Compound 1, wherein Compound 1 is present in an amount of 5% to 12% w/w, or from 8% to 12% w/w. In some embodiments, the pharmaceutical composition comprises 30 mg of Compound 1, wherein Compound 1 is present in an amount of 5% to 12% w/w, or for example 8% to 12% w/w.

In some embodiments, the pharmaceutical composition comprises 100 mg of Compound 1, wherein Compound 1 is present in an amount of about 12% w/w. In some embodiments, the pharmaceutical composition comprises 30 mg of Compound 1, wherein Compound 1 is present in an amount of about 12% w/w.

In some embodiments, the pharmaceutical composition comprises 100 mg of Compound 1, wherein Compound 1 is present in an amount of 12% w/w. In some embodiments, the pharmaceutical composition comprises 30 mg of Compound 1, wherein Compound 1 is present in an amount of 12% w/w.

In some embodiments, the pharmaceutical composition comprises 100 mg of Compound 1, wherein Compound 1 is present in an amount of about 8% w/w. In some embodiments, the pharmaceutical composition comprises 30 mg of Compound 1, wherein Compound 1 is present in an amount of about 8% w/w.

In some embodiments, the pharmaceutical composition comprises 100 mg of Compound 1, wherein Compound 1 is present in an amount of 8% w/w. In some embodiments, the pharmaceutical composition comprises 30 mg of Compound 1, wherein Compound 1 is present in an amount of 8% w/w.

### Excipients/Carriers

As discussed above, the pharmaceutical compositions disclosed herein comprise Compound 1 or a pharmaceutically acceptable salt thereof. The pharmaceutical compositions disclosed herein may further comprise pharmaceutical excipients such as diluents, binders, fillers, glidants, disintegrants, lubricants, solubilizers, and combinations thereof. Such compositions may be prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In some embodiments, the pharmaceutical composition comprises a diluent selected from the group consisting of dicalcium phosphate, cellulose, compressible sugars, dibasic calcium phosphate dehydrate, lactose, lactose monohydrate, mannitol, microcrystalline cellulose, starch, tribasic calcium phosphate, and combinations thereof.

In one embodiment, the pharmaceutical composition comprises lactose monohydrate in an amount ranging from about 0 to about 50% w/w, about 5% to about 45% w/w, about 10% to about 40% w/w, about 15% to about 35% w/w, or about 20% to about 30% w/w. In specific embodiments, the lactose monohydrate is present in the pharmaceutical composition at about 0% w/w, about 5% w/w, about 10% w/w, about 15% w/w, about 20% w/w, about 22% w/w, about 25% w/w, about 27% w/w, about 30% w/w, about 35% w/w, about 40% w/w, about 45% w/w, or about 50%. In one exemplary embodiment, lactose monohydrate is present in the pharmaceutical composition at about 22.3% w/w. In another exemplary embodiment, lactose monohydrate is present in the pharmaceutical composition at about 28% w/w. In yet another embodiment, lactose monohydrate is present in the pharmaceutical composition at about 20% w/w. In a further exemplary embodiment, lactose monohydrate is present in the pharmaceutical composition at about 24% w/w. In an additional exemplary embodiment, lactose monohydrate is present in the pharmaceutical composition at about 26% w/w. In another exemplary embodiment, lactose monohydrate is present in the pharmaceutical composition at about 30% w/w. In a further exemplary embodiment, lactose monohydrate is present in the pharmaceutical composition at about 30.8%.

In one embodiment, the pharmaceutical composition comprises lactose monohydrate in an amount ranging from 0 to 50% w/w, 5% to 45% w/w, 10% to 40% w/w, 15% to 35% w/w, or 20% to 30% w/w. In specific embodiments, the lactose monohydrate is present in the pharmaceutical composition at 0.1% w/w, 5% w/w, 10% w/w, 15% w/w, 20% w/w, 22% w/w, 25% w/w, 27% w/w, 30% w/w, 35% w/w, 40% w/w, 45% w/w, or 50%. In one exemplary embodiment, lactose monohydrate is present in the pharmaceutical composition at 22.3% w/w. In another exemplary embodiment, lactose monohydrate is present in the pharmaceutical composition at 28% w/w. In yet another embodiment, lactose monohydrate is present in the pharmaceutical composition at 20% w/w. In a further exemplary embodiment, lactose monohydrate is present in the pharmaceutical composition at 24% w/w. In an additional exemplary embodiment, lactose monohydrate is present in the pharmaceutical composition at 26% w/w. In another exemplary embodiment, lactose monohydrate is present in the pharmaceutical composition at 30% w/w. In a further exemplary embodiment, lactose monohydrate is present in the pharmaceutical composition at 30.8%.

In another embodiment, the pharmaceutical composition comprises microcrystalline cellulose in an amount ranging from about 0 to about 70% w/w, about 5% to about 65% w/w, about 10% to about 65% w/w, about 10% to about 60% w/w, about 15% to about 60% w/w, about 20% to about 60% w/w, or about 15% to about 60% w/w. In specific embodiments, the microcrystalline cellulose is present in the pharmaceutical composition at about 0% w/w, about 5% w/w, about 10% w/w, about 15% w/w, about 20% w/w, about 22% w/w, about 25% w/w, about 27% w/w, about 30% w/w, about 35% w/w, about 40% w/w, about 45% w/w, about 50% w/w, about 55% w/w, or about 60% w/w, or about 65% w/w. In one exemplary embodiment, microcrystalline cellulose is present in the pharmaceutical composition at about 27% w/w. In another exemplary embodiment, microcrystalline cellulose is present in the pharmaceutical composition at about 28.4% w/w. In yet another embodiment, microcrystalline cellulose is present in the pharmaceutical composition at about 45% w/w. In yet another embodiment, microcrystalline cellulose is present in the pharmaceutical composition at about 25.5% w/w. In yet another embodiment, microcrystalline cellulose is present in the pharmaceutical composition at about 62% w/w. In a further exemplary embodiment, microcrystalline cellulose is present in the pharmaceutical composition at about 57.5% w/w.

In another embodiment, the pharmaceutical composition comprises microcrystalline cellulose in an amount ranging from 0 to 70% w/w, 5% to 65% w/w, 10% to 65% w/w, 10% to 60% w/w, 15% to 60% w/w, 20% to 60% w/w, or 15% to 60% w/w. In specific embodiments, the microcrystalline cellulose is present in the pharmaceutical composition at 0.1% w/w, 5% w/w, 10% w/w, 15% w/w, 20% w/w, 22% w/w, 25% w/w, 27% w/w, 30% w/w, 35% w/w, 40% w/w, 45% w/w, 50% w/w, 55% w/w, 60% w/w, or 65% w/w. In one exemplary embodiment, microcrystalline cellulose is present in the pharmaceutical composition at 27% w/w. In another exemplary embodiment, microcrystalline cellulose is present in the pharmaceutical composition at 28.4% w/w. In yet another embodiment, microcrystalline cellulose is present in the pharmaceutical composition at 45% w/w. In yet another embodiment, microcrystalline cellulose is present in the pharmaceutical composition at 25.5% w/w. In yet another embodiment, microcrystalline cellulose is present in the pharmaceutical composition at 62% w/w. In a further exemplary embodiment, microcrystalline cellulose is present in the pharmaceutical composition at 57.5% w/w.

In one embodiment, the pharmaceutical composition comprises mannitol in an amount ranging from about 0 to about 70% w/w, about 10% to about 65% w/w, about 15% to about 65% w/w, about 15% to about 60% w/w, or about 20% to about 60% w/w. In specific embodiments, the mannitol is present in the pharmaceutical composition at about 0% w/w, about 5% w/w, about 10% w/w, about 15% w/w, about 20% w/w, about 22% w/w, about 25% w/w, about 27% w/w, about 30% w/w, about 35% w/w, about 40% w/w, about 45% w/w, about 50% w/w, about 55% w/w, about 57% w/w, about 60% w/w, or about 65% w/w. In one exemplary embodiment, mannitol is present in the pharmaceutical composition at about 54.6% w/w. In another exemplary embodiment, mannitol is present in the pharmaceutical composition at about 56.8% w/w. In yet another embodiment, mannitol is present in the pharmaceutical composition at about 51.4% w/w. In yet another embodiment, mannitol is present in the pharmaceutical composition at about 22.4% w/w. In a further exemplary embodiment, mannitol is present in the pharmaceutical composition at about 21.7% w/w.

In one embodiment, the pharmaceutical composition comprises mannitol in an amount ranging from 0 to 70% w/w, 10% to 65% w/w, 15% to 65% w/w, 15% to 60% w/w, or 20% to 60% w/w. In specific embodiments, the mannitol is present in the pharmaceutical composition at 0% w/w, 5% w/w, 10% w/w, 15% w/w, 20% w/w, 22% w/w, 25% w/w, 27% w/w, 30% w/w, 35% w/w, 40% w/w, 45% w/w, 50% w/w, 55% w/w, 57% w/w, 60% w/w, or 65% w/w. In one exemplary embodiment, mannitol is present in the pharmaceutical composition at 54.6% w/w. In another exemplary embodiment, mannitol is present in the pharmaceutical composition at 56.8% w/w. In yet another embodiment, mannitol is present in the pharmaceutical composition at 51.4% w/w. In yet another embodiment, mannitol is present in the pharmaceutical composition at 22.4% w/w. In a further exemplary embodiment, mannitol is present in the pharmaceutical composition at 21.7% w/w.

In yet another embodiment, the pharmaceutical composition comprises a mixture of lactose monohydrate and microcrystalline cellulose in an amount ranging from about 0 to about 95% w/w, about 20 to about 95% w/w, about 30 to about 95% w/w, about 40 to about 95% w/w, about 50% to about 95% w/w, about 55% to about 95% w/w, or about 60% to about 95% w/w. In specific embodiments, the mixture of lactose monohydrate and microcrystalline cellulose is present in the pharmaceutical composition at about 20% w/w, about 30% w/w, about 35% w/w, about 40% w/w, about 45% w/w, about 50% w/w, about 55% w/w, about 60% w/w, about 62%, about 65%, about 67%, about 70%, about 72%, about 75%, about 77%, about 80%, about 82%, about 85%, about 87%, about 90% w/w, or about 95% w/w.

In yet another embodiment, the pharmaceutical composition comprises a mixture of lactose monohydrate and microcrystalline cellulose in an amount ranging from 0 to 95% w/w, 20 to 95% w/w, 30 to 95% w/w, 40 to 95% w/w, 50% to 95% w/w, 55% to 95% w/w, or 60% to 95% w/w. In specific embodiments, the mixture of lactose monohydrate and microcrystalline cellulose is present in the pharmaceutical composition at 20% w/w, 30% w/w, 35% w/w, 40% w/w, 45% w/w, 50% w/w, 55% w/w, 60% w/w, 62%, 65%, 67%, 70%, 72%, 75%, 77%, 80%, 82%, 85%, 87%, 90% w/w, or 95% w/w.

In yet another embodiment, the pharmaceutical composition comprises a mixture of mannitol and microcrystalline cellulose in an amount ranging from about 0 to about 90% w/w, about 20 to about 90% w/w, about 30 to about 90% w/w, about 40 to about 90% w/w, about 50% to about 90% w/w, about 55% to about 90% w/w, or about 60% to about 90% w/w. In specific embodiments, the mixture of mannitol and microcrystalline cellulose is present in the pharmaceutical composition at about 20% w/w, about 30% w/w, about 35% w/w, about 40% w/w, about 45% w/w, about 50% w/w, about 55% w/w, about 60% w/w, about 62%, about 65%, about 67%, about 70%, about 72%, about 75%, about 77%, about 80%, about 81%, about 82%, about 83%, about 84%, about 85%, about 86%, about 87%, or about 90% w/w.

In yet another embodiment, the pharmaceutical composition comprises a mixture of mannitol and microcrystalline cellulose in an amount ranging from 0 to 90% w/w, 20 to 90% w/w, 30 to 90% w/w, 40 to 90% w/w, 50% to 90% w/w, 55% to 90% w/w, or 60% to 90% w/w. In specific embodiments, the mixture of mannitol and microcrystalline cellulose is present in the pharmaceutical composition at 20% w/w, 30% w/w, 35% w/w, 40% w/w, 45% w/w, 50% w/w, 55% w/w, 60% w/w, 62%, 65%, 67%, 70%, 72%, 75%, 77%, 80%, 81%, 82%, 83%, 84%, 85%, 86%, 87%, or 90% w/w.

In some embodiments, the pharmaceutical composition comprises a disintegrant selected from the group consisting of croscarmellose sodium, crospovidone, microcrystalline cellulose, modified corn starch, povidone, pregelatinized starch, sodium starch glycolate, and combinations thereof.

In one embodiment, the pharmaceutical composition comprises crospovidone in an amount ranging from about 1 to about 30% w/w, about 1 to about 25% w/w, about 1 to about 20% w/w, about 1 to about 15% w/w, about 2.5 to about 15% w/w, or about 5 to about 15% w/w. In specific embodiments, the crospovidone is present in the pharmaceutical composition in an amount of about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, or about 15% w/w. In one exemplary embodiment, the crospovidone is present in the pharmaceutical composition in an amount of about 7% w/w. In another exemplary embodiment, the crospovidone is present in the pharmaceutical composition in an amount of about 10% w/w. In yet another embodiment, the crospovidone is present in the pharmaceutical composition in an amount of about 5% w/w.

In one embodiment, the pharmaceutical composition comprises crospovidone in an amount ranging from 1 to 30% w/w, 1 to 25% w/w, 1 to 20% w/w, 1 to 15% w/w, 2.5 to 15% w/w, or 5 to 15% w/w. In specific embodiments, the crospovidone is present in the pharmaceutical composition in an amount of 1% w/w, 2% w/w, 3% w/w, 4% w/w, 5% w/w, 6% w/w, 7% w/w, 8% w/w, 9% w/w, 10% w/w, 11% w/w, 12% w/w, 13% w/w, 14% w/w, or 15% w/w. In one exemplary embodiment, the crospovidone is present in the pharmaceutical composition in an amount of 7% w/w. In another exemplary embodiment, the crospovidone is present in the pharmaceutical composition in an amount of 10% w/w. In yet another embodiment, the crospovidone is present in the pharmaceutical composition in an amount of 5% w/w.

In some embodiments, the pharmaceutical composition comprises a glidant selected from the group consisting of colloidal silicon dioxide, talc, starch, starch derivatives, and combinations thereof.

In one embodiment, the pharmaceutical composition comprises colloidal silicon dioxide in an amount ranging from about 0 to about 5% w/w, about 0.1 to about 4.5% w/w, about 0.1 to about 4% w/w, about 0.5 to about 5.0% w/w, about 0.5 to about 3% w/w, about 0.5 to about 2% w/w, or about 0.5 to about 1.5% w/w. In specific embodiments, the colloidal silicon dioxide is present in an amount of about 0% w/w, about 0.1% w/w, about 0.5% w/w, about 0.75% w/w, about 1% w/w, about 1.25% w/w, about 1.5% w/w, or about 2% w/w. In one exemplary embodiment, the colloidal silicon dioxide is present in the pharmaceutical composition in an amount of about 1% w/w.

In one embodiment, the pharmaceutical composition comprises colloidal silicon dioxide in an amount ranging from 0 to 5% w/w, 0.1 to 4.5% w/w, 0.1 to 4% w/w, 0.5 to 5.0% w/w, 0.5 to 3% w/w, 0.5 to 2% w/w, or 0.5 to 1.5% w/w. In specific embodiments, the colloidal silicon dioxide is present in an amount of 0% w/w, 0.1% w/w, 0.5% w/w, 0.75% w/w, 1% w/w, 1.25% w/w, 1.5% w/w, or 2% w/w. In one exemplary embodiment, the colloidal silicon dioxide is present in the pharmaceutical composition in an amount of 1% w/w.

In some embodiments, the pharmaceutical composition comprises a lubricant selected from the group consisting of calcium stearate, magnesium stearate, polyethylene glycol, sodium stearyl fumarate, stearic acid, talc, and combinations thereof.

In one embodiment, the pharmaceutical composition comprises magnesium stearate in an amount ranging from about 0 to about 3% w/w, about 0.1 to about 2.5% w/w, about 0.5 to about 3% w/w, about 0.5 to about 2.5% w/w, about 0.5 to about 2% w/w, about 1 to about 3% w/w, or from about 1 to about 2% w/w. In specific embodiments, the magnesium stearate is present in the pharmaceutical composition in an amount of about 0.1%, about 0.5% w/w, about 0.75% w/w, about 1% w/w, about 1.25% w/w, about 1.5% w/w, about 1.75% w/w, about 2% w/w, about 2.5% w/w, or about 3% w/w. In one exemplary embodiment, the magnesium stearate is present in the pharmaceutical composition in an amount of about 1.75% w/w. In another exemplary embodiment, the magnesium stearate is present in the pharmaceutical composition in an amount of about 1.5% w/w. In yet another embodiment, the magnesium stearate is present in the pharmaceutical composition in an amount of about 1% w/w.

In one embodiment, the pharmaceutical composition comprises magnesium stearate in an amount ranging from 0 to 3% w/w, 0.1 to 2.5% w/w, 0.5 to 3% w/w, 0.5 to 2.5% w/w, 0.5 to 2% w/w, 1 to 3% w/w, or from 1 to 2% w/w. In specific embodiments, the magnesium stearate is present in the pharmaceutical composition in an amount of 0.1%, 0.5% w/w, 0.75% w/w, 1% w/w, 1.25% w/w, 1.5% w/w, 1.75% w/w, 2% w/w, 2.5% w/w, or 3% w/w. In one exemplary embodiment, the magnesium stearate is present in the pharmaceutical composition in an amount of 1.75% w/w. In another exemplary embodiment, the magnesium stearate is present in the pharmaceutical composition in an amount of 1.5% w/w. In yet another embodiment, the magnesium stearate is present in the pharmaceutical composition in an amount of 1% w/w.

Some embodiments provided herein are directed to pharmaceutical compositions comprising: (a) about 5% to about 25% w/w of Compound 1 or a pharmaceutically acceptable salt thereof, (b) about 40% to about 60% w/w microcrystalline cellulose, (c) about 20% to about 30% w/w lactose monohydrate, (d) about 5% to about 10% w/w crospovidone, and about 1% to about 2% w/w magnesium stearate.

Some embodiments provided herein are directed to pharmaceutical compositions comprising: (a) 5% to 25% w/w of Compound 1 or a pharmaceutically acceptable salt thereof, (b) 40% to 60% w/w microcrystalline cellulose, (c) 20% to 30% w/w lactose monohydrate, (d) 5% to 10% w/w crospovidone, and 1% to 2% w/w magnesium stearate.

Some embodiments provided herein are directed to pharmaceutical compositions comprising: (a) about 0.5% to about 2% w/w of Compound 1 or a pharmaceutically acceptable salt thereof, (b) about 55% to about 65% w/w microcrystalline cellulose, (c) about 25% to about 35% w/w lactose monohydrate, (d) about 1% to about 10% w/w crospovidone, and about 0.5% to about 1.5% w/w magnesium stearate.

Some embodiments provided herein are directed to pharmaceutical compositions comprising: (a) 0.5% to 2% w/w of Compound 1 or a pharmaceutically acceptable salt thereof, (b) 55% to 65% w/w microcrystalline cellulose, (c) 25% to 35% w/w lactose monohydrate, (d) 1% to 10% w/w crospovidone, and 0.5% to 1.5% w/w magnesium stearate.

Some embodiments provided herein are directed to pharmaceutical compositions comprising: (a) about 20% to about 25% w/w of Compound 1 or a pharmaceutically acceptable salt thereof, (b) about 40% to about 50% w/w microcrystalline cellulose, (c) about 20% to about 30% w/w mannitol, (d) about 5% to about 10% w/w crospovidone, and about 1% to about 2% w/w magnesium stearate.

Some embodiments provided herein are directed to pharmaceutical compositions comprising: (a) 20% to 25% w/w of Compound 1 or a pharmaceutically acceptable salt thereof, (b) 40% to 50% w/w microcrystalline cellulose, (c) 20% to 30% w/w mannitol, (d) 5% to 10% w/w crospovidone, and 1% to 2% w/w magnesium stearate.

Some embodiments provided herein are directed to pharmaceutical compositions comprising: (a) about 5% to about 10% w/w of Compound 1 or a pharmaceutically acceptable salt thereof, (b) about 20% to about 30% w/w microcrystalline cellulose, (c) about 50% to about 60% w/w mannitol, (d) about 5% to about 10% w/w crospovidone, and about 1% to about 2% w/w magnesium stearate.

Some embodiments provided herein are directed to pharmaceutical compositions comprising: (a) 5% to 10% w/w of Compound 1 or a pharmaceutically acceptable salt thereof, (b) 20% to 30% w/w microcrystalline cellulose, (c) 50% to 60% w/w mannitol, (d) 5% to 10% w/w crospovidone, and 1% to 2% w/w magnesium stearate.

Some embodiments provided herein are directed to pharmaceutical compositions comprising: (a) about 5% to about 15% w/w of Compound 1 or a pharmaceutically acceptable salt thereof, (b) about 5% to about 10% w/w crospovidone, (c) about 50% to about 60% w/w mannitol, (d) about 20% to about 30% w/w microcrystalline cellulose, (e) about 1% to about 2% w/w magnesium stearate.

Some embodiments provided herein are directed to pharmaceutical compositions comprising: (a) 5% to 15% w/w of Compound 1 or a pharmaceutically acceptable salt thereof, (b) 5% to 10% w/w crospovidone, (c) 50% to 60% w/w mannitol, (d) 20% to 30% w/w microcrystalline cellulose, (e) 1% to 2% w/w magnesium stearate.

Some embodiments provided herein are directed to pharmaceutical compositions comprising: (a) about 10% w/w of Compound 1 or a pharmaceutically acceptable salt thereof, (b) about 7% w/w crospovidone, (c) about 55% w/w mannitol, (d) about 27% w/w microcrystalline cellulose, and (e) about 1.75% w/w magnesium stearate.

Some embodiments provided herein are directed to pharmaceutical compositions comprising: (a) 10% w/w of Compound 1 or a pharmaceutically acceptable salt thereof, (b) 7% w/w crospovidone, (c) 55% w/w mannitol, (d) 27% w/w microcrystalline cellulose, and (e) 1.75% w/w magnesium stearate.

Some embodiments provided herein are directed to pharmaceutical compositions comprising: (a) about 5% to about 15% w/w of Compound 1 or a pharmaceutically acceptable salt thereof, (b) about 5% to about 10% w/w crospovidone, (c) about 50% to about 60% w/w mannitol, (d) about 20% to about 30% w/w microcrystalline cellulose, and (e) about 1% to about 2% w/w magnesium stearate.

Some embodiments provided herein are directed to pharmaceutical compositions comprising: (a) 5% to 15% w/w of Compound 1 or a pharmaceutically acceptable salt thereof, (b) 5% to 10% w/w crospovidone, (c) 50% to 60% w/w mannitol, (d) 20% to 30% w/w microcrystalline cellulose, and (e) 1% to 2% w/w magnesium stearate.

Some embodiments provided herein are directed to pharmaceutical compositions comprising: (a) about 14% w/w of Compound 1 or a pharmaceutically acceptable salt thereof, (b) about 7% w/w crospovidone, (c) about 51% w/w mannitol, (d) about 25.5% w/w microcrystalline cellulose, and (e) about 1.75% w/w magnesium stearate.

Some embodiments provided herein are directed to pharmaceutical compositions comprising: (a) 14% w/w of Compound 1 or a pharmaceutically acceptable salt thereof, (b) 7% w/w crospovidone, (c) 51% w/w mannitol, (d) 25.5% w/w microcrystalline cellulose, and (e) 1.75% w/w magnesium stearate.

### Modes of Administration

The pharmaceutical compositions disclosed herein may be administered in either single or multiple doses by various methods including, for example, rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Another mode for administration is via inhalation. Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, the pharmaceutical compositions disclosed herein may be administered orally. Administration may be via, for example, tablet, capsule or enteric coated tablets. In making solid pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, sterile injectable solutions, and sterile packaged powders.

For preparing solid pharmaceutical compositions such as tablets, the principal active ingredient(s) may be mixed with a pharmaceutical excipient to form a solid preformulation composition containing a homogeneous mixture of the compounds described herein. When referring to these preformulation compositions as homogeneous, the active ingredient(s) may be dispersed evenly throughout the composition so that the composition may be readily subdivided into equally effective unit dosage forms such as tablets, pills and capsules.

In some embodiments, the pharmaceutical compositions disclosed herein can be formulated to provide quick, sustained or delayed release of the active ingredient(s) after administration to the subject by employing procedures known in the art. A “sustained release formulation” is a formulation which is designed to slowly release a therapeutic agent in the body over an extended period of time, whereas an “immediate release formulation” is a formulation which is designed to quickly release a therapeutic agent in the body over a shortened period of time. In some cases, the immediate release formulation may be coated such that the therapeutic agent is only released once it reached the desired target in the body (e.g., the stomach).

In some embodiments in which the pharmaceutical compositions disclosed herein are formulated into a tablet or pill, the tablet or pill may be coated or otherwise compounded to provide a dosage form affording the advantage of prolonged/sustained action, or to protect from the acid conditions of the stomach. For example, the tablet or pill may include a time-delay material such as glyceryl monostearate or glyceryl distearate employed alone or with a wax. Additionally, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for enteric layers or coatings, such materials including a number of polymeric acids and mixtures of polymeric acids with such materials as shellac, cetyl alcohol, and cellulose acetate.

In some embodiments in which the pharmaceutical compositions disclosed herein are formulated into a tablet or pill, the tablet or pill may be coated or otherwise compounded for immediate release.

In some embodiments in which the pharmaceutical compositions disclosed herein are formulated into a tablet or pill, the tablet or pill may have a film coating. In some embodiments, the film coating is configured to limit photolytic degradation. Suitable film coatings may be selected by routine screening of commercially available preparations. In one embodiment, the film coating comprises a polyvinyl alcohol-based coating. In another embodiment, the film coating comprises polyvinyl alcohol in combination with one or more of: titanium dioxide, polyethylene glycol, and talc. In yet another embodiment, the film coating is present in the pharmaceutical composition at about 3.0% w/w, or 3.0% w/w.

In some embodiments, the pharmaceutical compositions disclosed herein may be formulated as a monolayer tablet. Such a monolayer tablet may generally comprise the active ingredients (i.e., Compound 1 or an additional therapeutic agent as described herein) co-mixed in a single uniform layer. Exemplary methods for making monolayer tablets include, but are not limited to, co-dry granulation and bi-granulation. Co-dry granulation of the pharmaceutical compositions disclosed herein comprises dry granulating all the active ingredients (i.e., Compound 1 or an additional therapeutic agent as described herein) and excipients together. Bi-granulation of the pharmaceutical compositions disclosed herein is a multi-step process comprising (i) co-dry granulating two of the active ingredients (e.g., Compound 1 and an additional therapeutic agent as described herein) and excipients together to form granulation A, (ii) dry granulating the third active ingredient (e.g., another additional therapeutic agent as described herein) and excipients to form granulation B; and (iii) mixing/blending granulation A and granulation B together.

Some embodiments provided herein are directed to tablets comprising Compound 1 or a pharmaceutically acceptable salt thereof. In some embodiments, the pharmaceutically acceptable salt of Compound 1 is a tromethamine salt.

Some embodiments provided herein are directed to tablets comprising less than about 20% w/w of a Compound 1:

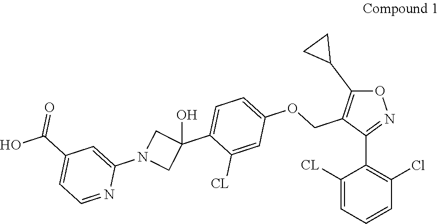

and at least one pharmaceutically acceptable carrier, and wherein the percentage by weight is relative to the total weight of the tablet.

Some embodiments provided herein are directed to tablets comprising 3% w/w to 20% w/w of a Compound 1:

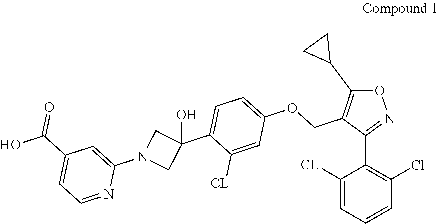

and at least one pharmaceutically acceptable carrier, and wherein the percentage by weight is relative to the total weight of the tablet.

Some embodiments provided herein are directed to tablets comprising less than about 25% w/w of a Compound 1:

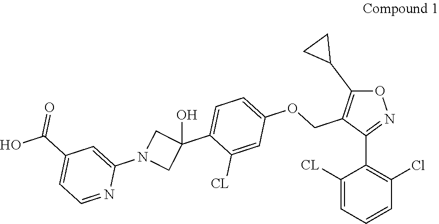

or a pharmaceutically acceptable salt thereof, and at least one pharmaceutically acceptable carrier, and wherein the percentage by weight is relative to the total weight of the tablet.

Some embodiments provided herein are directed to tablets comprising 3% w/w to 25% w/w of a Compound 1:

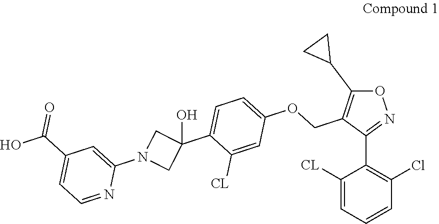

or a pharmaceutically acceptable salt thereof, and at least one pharmaceutically acceptable carrier, and wherein the percentage by weight is relative to the total weight of the tablet.

Some embodiments provided herein are directed to tablets comprising less than about 25% w/w of a tromethamine salt of Compound 1:

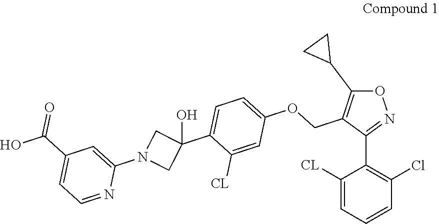

and at least one pharmaceutically acceptable carrier, and wherein the percentage by weight is relative to the total weight of the tablet. In some embodiments, the tablet comprises about 14% w/w of a tromethamine salt of Compound 1. In some embodiments, the tablet comprises about 6% w/w of a tromethamine salt of Compound 1.

Some embodiments provided herein are directed to tablets comprising 3% w/w to 20% w/w of a tromethamine salt of Compound 1:

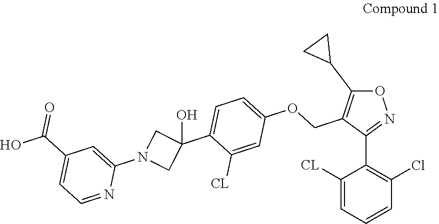

and at least one pharmaceutically acceptable carrier, and wherein the percentage by weight is relative to the total weight of the tablet. In some embodiments, the tablet comprises 10% to 14% w/w of a tromethamine salt of Compound 1. In some embodiments, the tablet comprises 6% w/w of a tromethamine salt of Compound 1.

In some embodiments, the tablet comprises about 1% w/w to about 25% w/w of Compound 1 or a pharmaceutically acceptable salt thereof. In some embodiments, the tablet comprises about 3% w/w to about 25% w/w of Compound 1 or a pharmaceutically acceptable salt thereof. In some embodiments, the tablet comprises about 5% w/w to about 25% w/w of Compound 1 or a pharmaceutically acceptable salt thereof. In some embodiments, the tablet comprises about 5% w/w to about 20% w/w of Compound 1 or a pharmaceutically acceptable salt thereof. In some embodiments, the tablet comprises about 5% w/w to about 15% w/w of Compound 1 or a pharmaceutically acceptable salt thereof. In some embodiments, the tablet comprises about 5% w/w to about 12% w/w of Compound 1 or a pharmaceutically acceptable salt thereof. In some embodiments, the tablet comprises about 5% w/w to about 10% w/w of Compound 1 or a pharmaceutically acceptable salt thereof. In some embodiments, the tablet comprises about 5% w/w to about 8% w/w of Compound 1 or a pharmaceutically acceptable salt thereof.

In some embodiments, the tablet comprises 1% w/w to 25% w/w of Compound 1 or a pharmaceutically acceptable salt thereof. In some embodiments, the tablet comprises 3% w/w to 25% w/w of Compound 1 or a pharmaceutically acceptable salt thereof. In some embodiments, the tablet comprises 5% w/w to 25% w/w of Compound 1 or a pharmaceutically acceptable salt thereof. In some embodiments, the tablet comprises 5% w/w to 20% w/w of Compound 1 or a pharmaceutically acceptable salt thereof. In some embodiments, the tablet comprises 5% w/w to 15% w/w of Compound 1 or a pharmaceutically acceptable salt thereof. In some embodiments, the tablet comprises 5% w/w to 12% w/w of Compound 1 or a pharmaceutically acceptable salt thereof. In some embodiments, the tablet comprises 8% w/w to 12% w/w of Compound 1 or a pharmaceutically acceptable salt thereof. In some embodiments, the tablet comprises 5% w/w to 10% w/w of Compound 1 or a pharmaceutically acceptable salt thereof. In some embodiments, the tablet comprises 5% w/w to 8% w/w of Compound 1 or a pharmaceutically acceptable salt thereof.

In some embodiments, the tablet comprises about 3% to about 25% w/w of a pharmaceutically acceptable salt of Compound 1. In some embodiments, the tablet comprises about 5% to about 25% w/w of a pharmaceutically acceptable salt of Compound 1.

In some embodiments, the tablet comprises 3% to 25% w/w of a pharmaceutically acceptable salt of Compound 1. In some embodiments, the tablet comprises 5% to 25% w/w of a pharmaceutically acceptable salt of Compound 1.

In some embodiments, the tablet comprises about 3% to about 25% w/w of a tromethamine salt of Compound 1. In some embodiments, the tablet comprises about 5% to about 25% w/w of a tromethamine salt of Compound 1.

In some embodiments, the tablet comprises 3% to 25% w/w of a tromethamine salt of Compound 1. In some embodiments, the tablet comprises 5% to 25% w/w of a tromethamine salt of Compound 1.

In some embodiments, the tablet comprises less than about 30% w/w of a pharmaceutically acceptable salt of Compound 1. In some embodiments, the tablet comprises less than about 25% w/w of a pharmaceutically acceptable salt of Compound 1. In some embodiments, the tablet comprises less than about 20% w/w of a pharmaceutically acceptable salt of Compound 1. In some embodiments, the tablet comprises less than about 18% w/w of a pharmaceutically acceptable salt of Compound 1. In some embodiments, the tablet comprises less than about 15% w/w of a pharmaceutically acceptable salt of Compound 1. In some embodiments, the tablet comprises less than about 10% w/w of a pharmaceutically acceptable salt of Compound 1. In some embodiments, the tablet comprises less than about 8% w/w of a pharmaceutically acceptable salt of Compound 1. In some embodiments, the tablet comprises less than about 7% w/w of a pharmaceutically acceptable salt of Compound 1. In some embodiments, the tablet comprises less than about 6% w/w of a pharmaceutically acceptable salt of Compound 1. In some embodiments, the tablet comprises less than about 5% w/w of a pharmaceutically acceptable salt of Compound 1. In some embodiments, the tablet comprises less than about 3% w/w of a pharmaceutically acceptable salt of Compound 1.

In some embodiments, the tablet comprises 1% to 20% w/w of a pharmaceutically acceptable salt of Compound 1. In some embodiments, the tablet comprises 1% to 18% w/w of a pharmaceutically acceptable salt of Compound 1. In some embodiments, the tablet comprises 1% to 15% w/w of a pharmaceutically acceptable salt of Compound 1. In some embodiments, the tablet comprises 1% to 10% w/w of a pharmaceutically acceptable salt of Compound 1. In some embodiments, the tablet comprises 1% to 8% w/w of a pharmaceutically acceptable salt of Compound 1. In some embodiments, the tablet comprises 1% to 7% w/w of a pharmaceutically acceptable salt of Compound 1. In some embodiments, the tablet comprises 1% to 6% w/w of a pharmaceutically acceptable salt of Compound 1. In some embodiments, the tablet comprises 5% w/w of a pharmaceutically acceptable salt of Compound 1. In some embodiments, the tablet comprises 1% to 3% w/w of a pharmaceutically acceptable salt of Compound 1.

In some embodiments, the tablet comprises less than about 30% w/w of a tromethamine salt of Compound 1. In some embodiments, the tablet comprises less than about 25% w/w of a tromethamine salt of Compound 1. In some embodiments, the tablet comprises less than about 20% w/w of a tromethamine salt of Compound 1. In some embodiments, the tablet comprises less than about 18% w/w of a tromethamine salt of Compound 1. In some embodiments, the tablet comprises less than about 15% w/w of a tromethamine salt of Compound 1. In some embodiments, the tablet comprises less than about 10% w/w of a tromethamine salt of Compound 1. In some embodiments, the tablet comprises less than about 8% w/w of a tromethamine salt of Compound 1. In some embodiments, the tablet comprises less than about 7% w/w of a tromethamine salt of Compound 1. In some embodiments, the tablet comprises less than about 6% w/w of a tromethamine salt of Compound 1. In some embodiments, the tablet comprises less than about 5% w/w of a tromethamine salt of Compound 1 .

In some embodiments, the tablet comprises 1% to 30% w/w of a tromethamine salt of Compound 1. In some embodiments, the tablet comprises 1% to 25% w/w of a tromethamine salt of Compound 1. In some embodiments, the tablet comprises 1% to 20% w/w of a tromethamine salt of Compound 1. In some embodiments, the tablet comprises 1% to 18% w/w of a tromethamine salt of Compound 1. In some embodiments, the tablet comprises 1% to 15% w/w of a tromethamine salt of Compound 1. In some embodiments, the tablet comprises 1% to 14% w/w of a tromethamine salt of Compound 1. In some embodiments, the tablet comprises 1% to 10% w/w of a tromethamine salt of Compound 1. In some embodiments, the tablet comprises 1% to 8% w/w of a tromethamine salt of Compound 1. In some embodiments, the tablet comprises 1% to 7% w/w of a tromethamine salt of Compound 1. In some embodiments, the tablet comprises 1% to 6% w/w of a tromethamine salt of Compound 1. In some embodiments, the tablet comprises 1% to 5% w/w of a tromethamine salt of Compound 1.

In some embodiments, the tablet comprises about 30% w/w of a pharmaceutically acceptable salt of Compound 1. In some embodiments, the tablet comprises about 25% w/w of a pharmaceutically acceptable salt of Compound 1. In some embodiments, the tablet comprises about 20% w/w of a pharmaceutically acceptable salt of Compound 1. In some embodiments, the tablet comprises about 18% w/w of a pharmaceutically acceptable salt of Compound 1. In some embodiments, the tablet comprises about 15% w/w of a pharmaceutically acceptable salt of Compound 1. In some embodiments, the tablet comprises about 14% w/w of a pharmaceutically acceptable salt of Compound 1. In some embodiments, the tablet comprises about 10% w/w of a pharmaceutically acceptable salt of Compound 1. In some embodiments, the tablet comprises about 8% w/w of a pharmaceutically acceptable salt of Compound 1. In some embodiments, the tablet comprises about 7% w/w of a pharmaceutically acceptable salt of Compound 1. In some embodiments, the tablet comprises about 6% w/w of a pharmaceutically acceptable salt of Compound 1. In some embodiments, the tablet comprises about 5% w/w of a pharmaceutically acceptable salt of Compound 1. In some embodiments, the tablet comprises about 1% w/w of a pharmaceutically acceptable salt of Compound 1.

In some embodiments, the tablet comprises 30% w/w of a pharmaceutically acceptable salt of Compound 1. In some embodiments, the tablet comprises 25% w/w of a pharmaceutically acceptable salt of Compound 1. In some embodiments, the tablet comprises 20% w/w of a pharmaceutically acceptable salt of Compound 1. In some embodiments, the tablet comprises 18% w/w of a pharmaceutically acceptable salt of Compound 1. In some embodiments, the tablet comprises 15% w/w of a pharmaceutically acceptable salt of Compound 1. In some embodiments, the tablet comprises 14% w/w of a pharmaceutically acceptable salt of Compound 1. In some embodiments, the tablet comprises 12% w/w of a pharmaceutically acceptable salt of Compound 1. In some embodiments, the tablet comprises 10% w/w of a pharmaceutically acceptable salt of Compound 1. In some embodiments, the tablet comprises 8% w/w of a pharmaceutically acceptable salt of Compound 1. In some embodiments, the tablet comprises 7% w/w of a pharmaceutically acceptable salt of Compound 1. In some embodiments, the tablet comprises 6% w/w of a pharmaceutically acceptable salt of Compound 1. In some embodiments, the tablet comprises 5% w/w of a pharmaceutically acceptable salt of Compound 1. In some embodiments, the tablet comprises 1% w/w of a pharmaceutically acceptable salt of Compound 1.

In some embodiments, the tablet comprises about 30% w/w of a tromethamine salt of Compound 1. In some embodiments, the tablet comprises about 25% w/w of a tromethamine salt of Compound 1. In some embodiments, the tablet comprises about 20% w/w of a tromethamine salt of Compound 1. In some embodiments, the tablet comprises about 18% w/w of a tromethamine salt of Compound 1. In some embodiments, the tablet comprises about 15% w/w of a tromethamine salt of Compound 1. In some embodiments, the tablet comprises about 14% w/w of a tromethamine salt of Compound 1. In some embodiments, the tablet comprises about 10% w/w of a tromethamine salt of Compound 1. In some embodiments, the tablet comprises about 8% w/w of a tromethamine salt of Compound 1. In some embodiments, the tablet comprises about 7% w/w of a tromethamine salt of Compound 1. In some embodiments, the tablet comprises about 6% w/w of a tromethamine salt of Compound 1. In some embodiments, the tablet comprises about 5% w/w of a tromethamine salt of Compound 1. In some embodiments, the tablet comprises about 1% w/w of a tromethamine salt of Compound 1.

In some embodiments, the tablet comprises 30% w/w of a tromethamine salt of Compound 1. In some embodiments, the tablet comprises 25% w/w of a tromethamine salt of Compound 1. In some embodiments, the tablet comprises 20% w/w of a tromethamine salt of Compound 1. In some embodiments, the tablet comprises 18% w/w of a tromethamine salt of Compound 1. In some embodiments, the tablet comprises 15% w/w of a tromethamine salt of Compound 1. In some embodiments, the tablet comprises 14% w/w of a tromethamine salt of Compound 1. In some embodiments, the tablet comprises 10% w/w of a tromethamine salt of Compound 1. In some embodiments, the tablet comprises 8% w/w of a tromethamine salt of Compound 1. In some embodiments, the tablet comprises 7% w/w of a tromethamine salt of Compound 1. In some embodiments, the tablet comprises 6% w/w of a tromethamine salt of Compound 1. In some embodiments, the tablet comprises 5% w/w of a tromethamine salt of Compound 1. In some embodiments, the tablet comprises 1% w/w of a tromethamine salt of Compound 1.

In some embodiments, the tablet comprises about 3% w/w to about 25% w/w of Compound 1. In some embodiments, the tablet comprises about 5% w/w to about 25% w/w of Compound 1. In some embodiments, the tablet comprises about 5% w/w to about 20% w/w of Compound 1. In some embodiments, the tablet comprises about 5% w/w to about 15% w/w of Compound 1. In some embodiments, the tablet comprises about 5% w/w to about 12% w/w of Compound 1. In some embodiments, the tablet comprises about 5% w/w to about 10% w/w of Compound 1. In some embodiments, the tablet comprises about 5% w/w to about 8% w/w of Compound 1.

In some embodiments, the tablet comprises 3% w/w to 20% w/w of Compound 1. In some embodiments, the tablet comprises 5% w/w to 20% w/w of Compound 1. In some embodiments, the tablet comprises 5% w/w to 15% w/w of Compound 1. In some embodiments, the tablet comprises 5% w/w to 12% w/w of Compound 1. In some embodiments, the tablet comprises 5% w/w to 10% w/w of Compound 1. In some embodiments, the tablet comprises 5% w/w to 8% w/w of Compound 1.

In some embodiments, the tablet comprises less than about 25% w/w of Compound 1. In some embodiments, the tablet comprises less than about 20% w/w of Compound 1. In some embodiments, the tablet comprises less than about 18% w/w of Compound 1. In some embodiments, the tablet comprises less than about 15% w/w of Compound 1. In some embodiments, the tablet comprises less than about 12% w/w of Compound 1. In some embodiments, the tablet comprises less than about 10% w/w of Compound 1. In some embodiments, the tablet comprises less than about 8% w/w of Compound 1. In some embodiments, the tablet comprises less than about 5% w/w of Compound 1.

In some embodiments, the tablet comprises 1% to 20% w/w of Compound 1. In some embodiments, the tablet comprises 1% to 18% w/w of Compound 1. In some embodiments, the tablet comprises 1% to 15% w/w of Compound 1. In some embodiments, the tablet comprises 1% to 12% w/w of Compound 1. In some embodiments, the tablet comprises 1% to 10% w/w of Compound 1. In some embodiments, the tablet comprises 1% to 8% w/w of Compound 1. In some embodiments, the tablet comprises 1% to 5% w/w of Compound 1.

In some embodiments, the tablet comprises about 20% w/w of Compound 1. In some embodiments, the tablet comprises about 18% w/w of Compound 1. In some embodiments, the tablet comprises about 15% w/w of Compound 1. In some embodiments, the tablet comprises about 12% w/w of Compound 1. In some embodiments, the tablet comprises about 10% w/w of Compound 1. In some embodiments, the tablet comprises about 8% w/w of Compound 1. In some embodiments, the tablet comprises about 5% w/w of Compound 1. In some embodiments, the tablet comprises about 2.5% w/w of Compound 1. In some embodiments, the tablet comprises about 1% w/w of Compound 1.

In some embodiments, the tablet comprises 20% w/w of Compound 1. In some embodiments, the tablet comprises 18% w/w of Compound 1. In some embodiments, the tablet comprises 15% w/w of Compound 1. In some embodiments, the tablet comprises 12% w/w of Compound 1. In some embodiments, the tablet comprises 10% w/w of Compound 1. In some embodiments, the tablet comprises 8% w/w of Compound 1. In some embodiments, the tablet comprises 5% w/w of Compound 1. In some embodiments, the tablet comprises 2.5% w/w of Compound 1. In some embodiments, the tablet comprises 1% w/w of Compound 1.

In some embodiments, the tablet comprises about 200 mg to about 1 mg of Compound 1. In some embodiments, the tablet comprises about 150 mg to about 10 mg of Compound 1. In some embodiments, the tablet comprises about 125 mg to about 15 mg of Compound 1. In some embodiments, the tablet comprises about 100 mg to about 30 mg of Compound 1. In some embodiments, the tablet comprises about 100 mg to about 20 mg of Compound 1. In some embodiments, the tablet comprises about 50 mg to about 200 mg of Compound 1. In some embodiments, the tablet comprises about 50 mg to about 150 mg of Compound 1. In some embodiments, the tablet comprises about 10 mg to about 50 mg of Compound 1.

In some embodiments, the tablet comprises 200 mg to 1 mg of Compound 1. In some embodiments, the tablet comprises 150 mg to 10 mg of Compound 1. In some embodiments, the tablet comprises 125 mg to 15 mg of Compound 1. In some embodiments, the tablet comprises 100 mg to 30 mg of Compound 1. In some embodiments, the tablet comprises 100 mg to 20 mg of Compound 1. In some embodiments, the tablet comprises 50 mg to 200 mg of Compound 1. In some embodiments, the tablet comprises 50 mg to 150 mg of Compound 1. In some embodiments, the tablet comprises 10 mg to 50 mg of Compound 1.

In some embodiments, the tablet comprises about 150 mg of Compound 1. In some embodiments, the tablet comprises about 100 mg of Compound 1. In some embodiments, the tablet comprises about 90 mg of Compound 1. In some embodiments, the tablet comprises about 80 mg of Compound 1. In some embodiments, the tablet comprises about 70 mg of Compound 1. In some embodiments, the tablet comprises about 60 mg of Compound 1. In some embodiments, the tablet comprises about 50 mg of Compound 1. In some embodiments, the tablet comprises about 40 mg of Compound 1. In some embodiments, the tablet comprises about 30 mg of Compound 1. In some embodiments, the tablet comprises about 20 mg of Compound 1. In some embodiments, the tablet comprises about 10 mg of Compound 1.

In some embodiments, the tablet comprises 150 mg of Compound 1. In some embodiments, the tablet comprises 100 mg of Compound 1. In some embodiments, the tablet comprises 90 mg of Compound 1. In some embodiments, the tablet comprises 80 mg of Compound 1. In some embodiments, the tablet comprises 70 mg of Compound 1. In some embodiments, the tablet comprises 60 mg of Compound 1. In some embodiments, the tablet comprises 50 mg of Compound 1. In some embodiments, the tablet comprises 40 mg of Compound 1. In some embodiments, the tablet comprises 30 mg of Compound 1. In some embodiments, the tablet comprises 20 mg of Compound 1. In some embodiments, the tablet comprises 10 mg of Compound 1.

In some embodiments, the tablet further comprises about 20% to about 70% w/w of microcrystalline cellulose. In some embodiments, the tablet further comprises about 25% to about 60% w/w of microcrystalline cellulose.

In some embodiments, the tablet further comprises 20% to 70% w/w of microcrystalline cellulose. In some embodiments, the tablet further comprises 25% to 60% w/w of microcrystalline cellulose.

In some embodiments, the tablet further comprises about 15% to about 65% w/w of lactose monohydrate, mannitol, or a combination thereof. In some embodiments, the tablet further comprises about 20% to about 60% w/w of lactose monohydrate, mannitol, or a combination thereof.

In some embodiments, the tablet further comprises 15% to 65% w/w of lactose monohydrate, mannitol, or a combination thereof. In some embodiments, the tablet further comprises 20% to 60% w/w of lactose monohydrate, mannitol, or a combination thereof.

In some embodiments, the tablet further comprises about 5% to about 10% w/w of crospovidone.

In some embodiments, the tablet further comprises 5% to 10% w/w of crospovidone.

In some embodiments, the tablet further comprises about 1% to about 2% w/w of magnesium stearate.

In some embodiments, the tablet further comprises 1% to 2% w/w of magnesium stearate.

Some embodiments provided herein are directed to tablets comprising: (a) about 5% to about 25% w/w of Compound 1 or a pharmaceutically acceptable salt thereof, (b) about 40% to about 60% w/w microcrystalline cellulose, (c) about 20% to about 30% w/w lactose monohydrate, (d) about 5% to about 10% w/w crospovidone, and (e) about 1% to about 2% w/w magnesium stearate.

Some embodiments provided herein are directed to tablets comprising: (a) 5% to 25% w/w of Compound 1 or a pharmaceutically acceptable salt thereof, (b) 40% to 60% w/w microcrystalline cellulose, (c) 20% to 30% w/w lactose monohydrate, (d) 5% to 10% w/w crospovidone, and (e) 1% to 2% w/w magnesium stearate.

In some embodiments, a tablet comprises (a) about 5% to about 10% w/w of Compound 1 or a pharmaceutically acceptable salt thereof, (b) about 40% to about 60% w/w microcrystalline cellulose, (c) about 20% to about 30% w/w lactose monohydrate, (d) about 5% to about 10% w/w crospovidone, and (e) about 1% to about 2% w/w magnesium stearate.

In some embodiments, a tablet comprises (a) 5% to 10% w/w of Compound 1 or a pharmaceutically acceptable salt thereof, (b) 40% to 60% w/w microcrystalline cellulose, (c) 20% to 30% w/w lactose monohydrate, (d) 5% to 10% w/w crospovidone, and (e) 1% to 2% w/w magnesium stearate.

In some embodiments, a tablet comprises (a) about 6% w/w of Compound 1 or a pharmaceutically acceptable salt thereof, (b) about 58% w/w microcrystalline cellulose, (c) about 28% w/w lactose monohydrate, (d) about 7% w/w crospovidone, and (e) about 1.5% w/w magnesium stearate.

In some embodiments, a tablet comprises (a) 6% w/w of Compound 1 or a pharmaceutically acceptable salt thereof, (b) 58% w/w microcrystalline cellulose, (c) 28% w/w lactose monohydrate, (d) 7% w/w crospovidone, and (e) 1.5% w/w magnesium stearate.

In some embodiments, a tablet comprises (a) about 5% w/w of Compound 1, (b) about 58% w/w microcrystalline cellulose, (c) about 28% w/w lactose monohydrate, (d) about 7% w/w crospovidone, and (e) about 1.5% w/w magnesium stearate.

In some embodiments, a tablet comprises (a) 5% w/w of Compound 1, (b) 58% w/w microcrystalline cellulose, (c) 28% w/w lactose monohydrate, (d) 7% w/w crospovidone, and (e) 1.5% w/w magnesium stearate.

In some embodiments, a tablet comprises (a) about 20% to about 25% w/w of Compound 1 or a pharmaceutically acceptable salt thereof, (b) about 40% to about 50% w/w microcrystalline cellulose, (c) about 20% to about 30% w/w lactose monohydrate, (d) about 5% to about 10% w/w crospovidone, and (e) about 1% to about 2% w/w magnesium stearate.

In some embodiments, a tablet comprises (a) 20% to 25% w/w of Compound 1 or a pharmaceutically acceptable salt thereof, (b) 40% to 50% w/w microcrystalline cellulose, (c) 20% to 30% w/w lactose monohydrate, (d) 5% to 10% w/w crospovidone, and (e) 1% to 2% w/w magnesium stearate.

In some embodiments, a tablet comprises (a) about 24% w/w of Compound 1 or a pharmaceutically acceptable salt thereof, (b) about 45% w/w microcrystalline cellulose, (c) about 22% w/w lactose monohydrate, (d) about 7% w/w crospovidone, and (e) about 1.5% w/w magnesium stearate.

In some embodiments, a tablet comprises (a) 24% w/w of Compound 1 or a pharmaceutically acceptable salt thereof, (b) 45% w/w microcrystalline cellulose, (c) 22% w/w lactose monohydrate, (d) 7% w/w crospovidone, and (e) 1.5% w/w magnesium stearate.

In some embodiments, a tablet comprises (a) about 20% w/w of Compound 1, (b) about 45% w/w microcrystalline cellulose, (c) about 22% w/w lactose monohydrate, (d) about 7% w/w crospovidone, and (e) about 1.5% w/w magnesium stearate.

In some embodiments, a tablet comprises (a) 20% w/w of Compound 1, (b) 45% w/w microcrystalline cellulose, (c) 22% w/w lactose monohydrate, (d) 7% w/w crospovidone, and (e) 1.5% w/w magnesium stearate.

In some embodiments, a tablet comprises: (a) about 0.5% to about 2% w/w of Compound 1 or a pharmaceutically acceptable salt thereof, (b) about 55% to about 65% w/w microcrystalline cellulose, (c) about 25% to about 35% w/w lactose monohydrate, (d) about 1% to about 10% w/w crospovidone, and (e) about 0.5% to about 1.5% w/w magnesium stearate.

In some embodiments, a tablet comprises: (a) 0.5% to 2% w/w of Compound 1 or a pharmaceutically acceptable salt thereof, (b) 55% to 65% w/w microcrystalline cellulose, (c) 25% to 35% w/w lactose monohydrate, (d) 1% to 10% w/w crospovidone, and (e) 0.5% to 1.5% w/w magnesium stearate.

In some embodiments, a tablet comprises: (a) about 1% w/w of Compound 1 or a pharmaceutically acceptable salt thereof, (b) about 62% w/w microcrystalline cellulose, (c) about 31% w/w lactose monohydrate, (d) about 5% w/w crospovidone, and (e) about 1% w/w magnesium stearate.

In some embodiments, a tablet comprises: (a) 1% w/w of Compound 1 or a pharmaceutically acceptable salt thereof, (b) 62% w/w microcrystalline cellulose, (c) 31% w/w lactose monohydrate, (d) 5% w/w crospovidone, and (e) 1% w/w magnesium stearate.

In some embodiments, a tablet comprises: (a) about 1% w/w of Compound 1, (b) about 62% w/w microcrystalline cellulose, (c) about 31% w/w lactose monohydrate, (d) about 5% w/w crospovidone, and (e) about 1% w/w magnesium stearate.

In some embodiments, a tablet comprises: (a) 1% w/w of Compound 1, (b) 62% w/w microcrystalline cellulose, (c) 31% w/w lactose monohydrate, (d) 5% w/w crospovidone, and (e) 1% w/w magnesium stearate.

Some embodiments provided herein are directed to tablets comprising: (a) about 20% to about 25% w/w of Compound 1 or a pharmaceutically acceptable salt thereof, (b) about 40% to about 50% w/w microcrystalline cellulose, (c) about 20% to about 30% w/w mannitol, (d) about 5% to about 10% w/w crospovidone, and (e) about 1% to about 2% w/w magnesium stearate.

Some embodiments provided herein are directed to tablets comprising: (a) 20% to 25% w/w of Compound 1 or a pharmaceutically acceptable salt thereof, (b) 40% to 50% w/w microcrystalline cellulose, (c) 20% to 30% w/w mannitol, (d) 5% to 10% w/w crospovidone, and (e) 1% to 2% w/w magnesium stearate.

Some embodiments provided herein are directed to tablets comprising: (a) about 24% w/w of Compound 1 or a pharmaceutically acceptable salt thereof, (b) about 45% w/w microcrystalline cellulose, (c) about 22% w/w mannitol, (d) about 7% w/w crospovidone, and about (e) 1.5% w/w magnesium stearate.

Some embodiments provided herein are directed to tablets comprising: (a) 24% w/w of Compound 1 or a pharmaceutically acceptable salt thereof, (b) 45% w/w microcrystalline cellulose, (c) 22% w/w mannitol, (d) 7% w/w crospovidone, and (e) 1.5% w/w magnesium stearate.

Some embodiments provided herein are directed to tablets comprising: (a) about 20% w/w of Compound 1, (b) about 45% w/w microcrystalline cellulose, (c) about 22% w/w mannitol, (d) about 7% w/w crospovidone, and (e) about 1.5% w/w magnesium stearate.

Some embodiments provided herein are directed to tablets comprising: (a) 20% w/w of Compound 1, (b) 45% w/w microcrystalline cellulose, (c) 22% w/w mannitol, (d) 7% w/w crospovidone, and (e) 1.5% w/w magnesium stearate.

Some embodiments provided herein are directed to tablets comprising: (a) about 5% to about 10% w/w of Compound 1 or a pharmaceutically acceptable salt thereof, (b) about 20% to about 30% w/w microcrystalline cellulose, (c) about 50% to about 60% w/w mannitol, (d) about 5% to about 10% w/w crospovidone, and (e) about 1% to about 2% w/w magnesium stearate.

Some embodiments provided herein are directed to tablets comprising: (a) 5% to 10% w/w of Compound 1 or a pharmaceutically acceptable salt thereof, (b) 20% to 30% w/w microcrystalline cellulose, (c) 50% to 60% w/w mannitol, (d) 5% to 10% w/w crospovidone, and (e) 1% to 2% w/w magnesium stearate.

Some embodiments provided herein are directed to tablets comprising: (a) about 6% w/w of Compound 1 or a pharmaceutically acceptable salt thereof, (b) about 28% w/w microcrystalline cellulose, (c) about 57% w/w mannitol, (d) about 7% w/w crospovidone, and (e) about 1.75% w/w magnesium stearate.

Some embodiments provided herein are directed to tablets comprising: (a) 6% w/w of Compound 1 or a pharmaceutically acceptable salt thereof, (b) 28% w/w microcrystalline cellulose, (c) 57% w/w mannitol, (d) 7% w/w crospovidone, and (e) 1.75% w/w magnesium stearate.

Some embodiments provided herein are directed to tablets comprising: (a) about 5% w/w of Compound 1, (b) about 28% w/w microcrystalline cellulose, (c) about 57% w/w mannitol, (d) about 7% w/w crospovidone, and (e) about 1.75% w/w magnesium stearate.

Some embodiments provided herein are directed to tablets comprising: (a) 5% w/w of Compound 1, (b) 28% w/w microcrystalline cellulose, (c) 57% w/w mannitol, (d) 7% w/w crospovidone, and (e) 1.75% w/w magnesium stearate.

Some embodiments provided herein are directed to tablets comprising: (a) about 5% to about 15% w/w of Compound 1 or a pharmaceutically acceptable salt thereof, (b) about 5% to about 10% w/w crospovidone, (c) about 50% to about 60% w/w mannitol, (d) about 20% to about 30% w/w microcrystalline cellulose, and (e) about 1% to about 2% w/w magnesium stearate.

Some embodiments provided herein are directed to tablets comprising: (a) 5% to 15% w/w of Compound 1 or a pharmaceutically acceptable salt thereof, (b) 5% to 10% w/w crospovidone, (c) 50% to 60% w/w mannitol, (d) 20% to 30% w/w microcrystalline cellulose, and (e) 1% to 2% w/w magnesium stearate.

Some embodiments provided herein are directed to tablets comprising: (a) about 10% w/w of Compound 1 or a pharmaceutically acceptable salt thereof, (b) about 7% w/w crospovidone, (c) about 55% w/w mannitol, (d) about 27% w/w microcrystalline cellulose, and (e) about 1.75% w/w magnesium stearate.

Some embodiments provided herein are directed to tablets comprising: (a) 10% w/w of Compound 1 or a pharmaceutically acceptable salt thereof, (b) 7% w/w crospovidone, (c) 55% w/w mannitol, (d) 27% w/w microcrystalline cellulose, and (e) 1.75% w/w magnesium stearate.

Some embodiments provided herein are directed to tablets comprising: (a) about 8% w/w of Compound 1, (b) about 7% w/w crospovidone, (c) about 55% w/w mannitol, (d) about 27% w/w microcrystalline cellulose, and (e) about 1.75% w/w magnesium stearate.

Some embodiments provided herein are directed to tablets comprising: (a) 8% w/w of Compound 1, (b) 7% w/w crospovidone, (c) 55% w/w mannitol, (d) 27% w/w microcrystalline cellulose, and (e) 1.75% w/w magnesium stearate.

Some embodiments provided herein are directed to tablets comprising: (a) about 5% to about 15% w/w of Compound 1 or a pharmaceutically acceptable salt thereof, (b) about 5% to about 10% w/w crospovidone, (c) about 50% to about 60% w/w mannitol, (d) about 20% to about 30% w/w microcrystalline cellulose, and (e) about 1% to about 2% w/w magnesium stearate.

Some embodiments provided herein are directed to tablets comprising: (a) 5% to 15% w/w of Compound 1 or a pharmaceutically acceptable salt thereof, (b) 5% to 10% w/w crospovidone, (c) 50% to 60% w/w mannitol, (d) 20% to 30% w/w microcrystalline cellulose, and (e) 1% to 2% w/w magnesium stearate.

Some embodiments provided herein are directed to tablets comprising: (a) about 14% w/w of Compound 1 or a pharmaceutically acceptable salt thereof, (b) about 7% w/w crospovidone, (c) about 51% w/w mannitol, (d) about 25.5% w/w microcrystalline cellulose, and (e) about 1.75% w/w magnesium stearate.

Some embodiments provided herein are directed to tablets comprising: (a) 14% w/w of Compound 1 or a pharmaceutically acceptable salt thereof, (b) 7% w/w crospovidone, (c) 51% w/w mannitol, (d) 25.5% w/w microcrystalline cellulose, and (e) 1.75% w/w magnesium stearate.

Some embodiments provided herein are directed to tablets comprising: (a) about 5% to about 15% w/w of Compound 1, (b) about 5% to about 10% w/w crospovidone, (c) about 50% to about 60% w/w mannitol, (d) about 20% to about 30% w/w microcrystalline cellulose, and (e) about 1% to about 2% w/w magnesium stearate.

Some embodiments provided herein are directed to tablets comprising: (a) 5% to 15% w/w of Compound 1, (b) 5% to 10% w/w crospovidone, (c) 50% to 60% w/w mannitol, (d) 20% to 30% w/w microcrystalline cellulose, and (e) 1% to 2% w/w magnesium stearate.

Some embodiments provided herein are directed to tablets comprising: (a) about 12% w/w of Compound 1, (b) about 7% w/w crospovidone, (c) about 51% w/w mannitol, (d) about 25.5% w/w microcrystalline cellulose, and (e) about 1.75% w/w magnesium stearate.

Some embodiments provided herein are directed to tablets comprising: (a) 12% w/w of Compound 1, (b) 7% w/w crospovidone, (c) 51% w/w mannitol, (d) 25.5% w/w microcrystalline cellulose, and (e) 1.75% w/w magnesium stearate.

In some embodiments, the tablet is a film-coated tablet.

In some embodiments, the tablet further comprises selonsertib.

In some embodiments, the tablet further comprises firsocostat.

### Dosing

The effective dosage of active ingredient employed may vary depending on the particular compound employed, the mode of administration, the condition being treated and the severity of the condition being treated. Such dosage may be ascertained readily by a person skilled in the art.

When treating or preventing FXR mediated conditions for which compounds of the present disclosure are indicated, generally satisfactory results are obtained when the compounds of the present disclosure are administered at a daily dosage of from about 0.1 milligram to about 100 milligram per kilogram of animal body weight. In some embodiments, the compounds of the present disclosure are given as a single daily dose or in divided doses two to six times a day, or in sustained release form. For most large mammals, the total daily dosage is from about 1 milligram to about 1000 milligrams, or from about 1 milligram to about 50 milligrams. In the case of a 70 kg adult human, the total daily dose will generally be from about 7 milligrams to about 350 milligrams. This dosage regimen may be adjusted to provide the optimal therapeutic response. In some embodiments, the total daily dosage is from about 1 milligram to about 900 milligrams, about 10 milligrams to about 800 milligrams, about 20 milligrams to about 700 milligrams, about 30 milligrams to about 600 milligrams, about 40 milligrams to about 550 milligrams, or about 50 milligrams to about 400 milligrams. In certain embodiments, compounds of the present disclosure are administered at a daily dosage of from 0.1 milligram to 100 milligram per kilogram of animal body weight. In some embodiments, the compounds of the present disclosure are given as a single daily dose or in divided doses two to six times a day, or in sustained release form. For most large mammals, the total daily dosage is from 1 milligram to 1000 milligrams, or from 1 milligram to 50 milligrams. In the case of a 70 kg adult human, the total daily dose will generally be from 7 milligrams to 350 milligrams. This dosage regimen may be adjusted to provide the optimal therapeutic response. In some embodiments, the total daily dosage is from 1 milligram to 900 milligrams, 10 milligrams to 800 milligrams, 20 milligrams to 700 milligrams, 30 milligrams to 600 milligrams, 40 milligrams to 550 milligrams, or 50 milligrams to 400 milligrams.

The compounds of the present application or the compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy and are frequently alternated with resting periods of about 1 to 28 days, commonly about 7 days or about 14 days, between cycles. The treatment cycles, in other embodiments, may also be continuous. In some embodiments, administration or treatment with the compounds may be continued for a number of days; for example, commonly treatment would continue for 7 to 28 days, 14 days, or 28 days, for one cycle of treatment. Treatment cycles are well known in cancer chemotherapy and are frequently alternated with resting periods of 1 to 28 days, 7 days or 14 days, between cycles. The treatment cycles, in other embodiments, may also be continuous.

In a particular embodiment, the methods provided herein comprise administering to the subject an initial daily dose of about 1 to 800 mg, or 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

In some embodiments, the methods provided herein comprise administering to the subject a daily dosage of about 100 mg of Compound 1.

In some embodiments, the methods provided herein comprise administering to the subject a daily dosage of 100 mg of Compound 1.

In some embodiments, the methods provided herein comprise administering to the subject a daily dosage of about 30 mg of Compound 1.

In some embodiments, the methods provided herein comprise administering to the subject a daily dosage of 30 mg of Compound 1.

### Treatment Methods and Uses

“Treatment” or “treating” is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition); (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

The disclosure further relates to the use of compounds described herein and compositions described herein for the treatment and/or prophylaxis of diseases and/or conditions through binding of said nuclear receptor by said compounds. Further the present disclosure relates to the use of compounds described herein and compositions described herein for the preparation of a medicament for the treatment and/or prophylaxis of diseases and/or conditions through binding of said nuclear receptor by said compounds.

Also provided herein are methods of treating a patient having a FXR mediated condition. In some embodiments, the method includes administering a compound or composition disclosed herein. In some embodiments, a method of treating a patient having an FXR mediated condition comprises administering a pharmaceutical composition described herein. In some embodiments, a method of treating a patient having an FXR mediated condition comprises administering a tablet described herein.

Also provided herein are methods of treating or preventing a disease or condition in a patient in need thereof, comprising administering a pharmaceutical composition described herein, wherein the disease or condition is congenital hepatic fibrosis.

In some embodiments, a method of treating a patient having congenital hepatic fibrosis comprises administering a pharmaceutical composition comprising Compound 1 as described herein. In some embodiments, a method of treating a patient having congenital hepatic fibrosis comprises administering a tablet comprising Compound 1 as described herein.

In some embodiments, a compound or composition disclosed herein is provided for use in the treatment of a FXR mediated condition.

In some embodiments, a compound or composition disclosed herein is provided for the manufacture of a medicament for the treatment of a FXR mediated condition.

In some embodiments, the FXR mediated condition is: a chronic intrahepatic or some form of extrahepatic cholestatic condition; liver fibrosis; an obstructive inflammatory disorder of the liver; chronic inflammatory disorder of the liver; liver cirrhosis; liver steatosis or an associated syndrome; cholestatic or fibrotic effects that are associated with alcohol-induced cirrhosis or with viral-borne forms of hepatitis; liver failure or liver ischemia after major liver resection; chemotherapy associated steatohepatitis (CASH); acute liver failure; or Inflammatory Bowel Disease.

In some embodiments, the FXR mediated condition is a lipid and lipoprotein disorder; Type I Diabetes; Type II Diabetes; clinical complications of Type I and Type II Diabetes selected from the group consisting of diabetic nephropathy, diabetic neuropathy, diabetic retinopathy and other observed effects of clinically manifest long term Diabetes; Non-Alcoholic Fatty Liver Disease (NAFLD); Non-Alcoholic Steatohepatitis (NASH); obesity; a metabolic syndrome selected from the group consisting of combined conditions of dyslipidemia, diabetes and abnormally high body-mass index; acute myocardial infarction; acute stroke; or thrombosis which occurs as an endpoint of chronic obstructive atherosclerosis.

In some embodiments, the FXR mediated condition is: a non-malignant hyperproliferative disorder; and a malignant hyperproliferative disorder selected from the group consisting of hepatocellular carcinoma, colon adenoma, and polyposis; colon adenocarcinoma; breast cancer; pancreas adenocarcinoma; Barrett's esophagus; or other forms of neoplastic diseases of the gastrointestinal tract and the liver.

In some embodiments, the FXR mediated condition is Non-Alcoholic Steatohepatitis (NASH), primary sclerosing cholangitis (PSC), or primary biliary cirrhosis (PBC).

In some embodiments, the FXR mediated condition is congenital hepatic fibrosis. In some embodiments, the FXR mediated condition is NASH. In some embodiments, the FXR mediated condition is PSC.

In some embodiments, the present disclosure relates to the use of compounds and compositions disclosed herein in the preparation of a medicament for the prophylaxis and/or treatment of chronic intrahepatic or some forms of extrahepatic cholestatic conditions, of liver fibrosis, of acute intrahepatic cholestatic conditions, of obstructive or chronic inflammatory disorders that arise out of improper bile composition, of gastrointestinal conditions with a reduced uptake of dietary fat and fat-soluble dietary vitamins, of inflammatory bowel diseases, of lipid and lipoprotein disorders, of Type II Diabetes and clinical complications of Type I and Type II Diabetes, of conditions and diseases which result from chronic fatty and fibrotic degeneration of organs due to enforced lipid and specifically triglyceride accumulation and subsequent activation of profibrotic pathways, of obesity and metabolic syndrome (combined conditions of dyslipidemia, diabetes and abnormally high body-mass index), of acute myocardial infarction, of acute stroke, of thrombosis which occurs as an endpoint of chronic obstructive atherosclerosis, of persistent infections by intracellular bacteria or parasitic protozoa, of non-malignant hyperproliferative disorders, of malignant hyperproliferative disorders, of colon adenocarcinoma and hepatocellular carcinoma in particular, of liver steatosis and associated syndromes, of liver failure or liver malfunction as an outcome of chronic liver diseases or of surgical liver resection, of Hepatitis B infection, of Hepatitis C infection, of cholestatic and fibrotic effects that are associated with alcohol-induced cirrhosis or with viral-borne forms of hepatitis, and/or of congenital hepatic fibrosis.

Some embodiments provided herein are directed to a method of treating a condition mediated by FXR in a patient in need thereof comprising administering a pharmaceutical composition comprising less than about 20% w/w of a Compound 1, and at least one pharmaceutically acceptable carrier, and wherein the percentage by weight is relative to the total weight of the pharmaceutical composition.

Some embodiments provided herein are directed to a method of treating a condition mediated by FXR in a patient in need thereof comprising administering a pharmaceutical composition comprising 1% to 20% w/w of a Compound 1, and at least one pharmaceutically acceptable carrier, and wherein the percentage by weight is relative to the total weight of the pharmaceutical composition.

Some embodiments provided herein are directed to a method of treating a condition mediated by FXR in a patient in need thereof comprising administering a pharmaceutical composition comprising less than about 25% w/w of a Compound 1, or a pharmaceutically acceptable salt thereof, and at least one pharmaceutically acceptable carrier, and wherein the percentage by weight is relative to the total weight of the pharmaceutical composition.

Some embodiments provided herein are directed to a method of treating a condition mediated by FXR in a patient in need thereof comprising administering a pharmaceutical composition comprising 1% to 25% w/w of a Compound 1, or a pharmaceutically acceptable salt thereof, and at least one pharmaceutically acceptable carrier, and wherein the percentage by weight is relative to the total weight of the pharmaceutical composition.

Some embodiments provided herein are directed to a method of treating a condition mediated by FXR in a patient in need thereof comprising administering a tablet comprising less than about 20% w/w of a Compound 1, and at least one pharmaceutically acceptable carrier, and wherein the percentage by weight is relative to the total weight of the tablet.

Some embodiments provided herein are directed to a method of treating a condition mediated by FXR in a patient in need thereof comprising administering a tablet comprising 1% to 20% w/w of a Compound 1, and at least one pharmaceutically acceptable carrier, and wherein the percentage by weight is relative to the total weight of the tablet.

Some embodiments provided herein are directed to a method of treating a condition mediated by FXR in a patient in need thereof comprising administering a tablet comprising less than about 25% w/w of a Compound 1, or a pharmaceutically acceptable salt thereof, and at least one pharmaceutically acceptable carrier, and wherein the percentage by weight is relative to the total weight of the tablet.

Some embodiments provided herein are directed to a method of treating a condition mediated by FXR in a patient in need thereof comprising administering a tablet comprising 1% to 25% w/w of a Compound 1, or a pharmaceutically acceptable salt thereof, and at least one pharmaceutically acceptable carrier, and wherein the percentage by weight is relative to the total weight of the tablet.

In some embodiments, the condition mediated by FXR is Non-Alcoholic Steatohepatitis (NASH). In such embodiments, a tablet as described herein comprises about 1 mg to about 200 mg, or from about 30 mg to about 100 mg of Compound 1. For example, in some embodiments, a tablet as described herein comprises about 30 mg or about 100 mg of Compound 1. In some embodiments, a tablet as described herein comprises about 1 mg or about 200 mg of Compound 1.

In some embodiments, the condition mediated by FXR is Non-Alcoholic Steatohepatitis (NASH). In such embodiments, a tablet as described herein comprises 1 mg to 200 mg, or from 30 mg to 100 mg of Compound 1. For example, in some embodiments, a tablet as described herein comprises 30 mg or 100 mg of Compound 1. In some embodiments, a tablet as described herein comprises 1 mg or 200 mg of Compound 1.

In some embodiments, the condition mediated by FXR is primary sclerosing cholangitis (PSC). In such embodiments, a tablet as described herein comprises about 1 mg to about 200 mg, or from about 30 mg to about 100 mg of Compound 1. For example, in some embodiments, a tablet as described herein comprises about 30 mg or about 100 mg of Compound 1. In some embodiments, a tablet as described herein comprises about 1 mg or about 200 mg of Compound 1.

In some embodiments, the condition mediated by FXR is primary sclerosing cholangitis (PSC). In such embodiments, a tablet as described herein comprises 1 mg to 200 mg, or from 30 mg to 100 mg of Compound 1. For example, in some embodiments, a tablet as described herein comprises 30 mg or 100 mg of Compound 1. In some embodiments, a tablet as described herein comprises 1 mg or 200 mg of Compound 1.

In some embodiments, the condition mediated by FXR is primary biliary cirrhosis (PBC). In such embodiments, a tablet as described herein comprises about 1 mg to about 200 mg, or from about 30 mg to about 100 mg of Compound 1. For example, in some embodiments, a tablet as described herein comprises about 30 mg or about 100 mg of Compound 1. In some embodiments, a tablet as described herein comprises about 1 mg or about 200 mg of Compound 1.

In some embodiments, the condition mediated by FXR is primary biliary cirrhosis (PBC). In such embodiments, a tablet as described herein comprises 1 mg to 200 mg, or from 30 mg to 100 mg of Compound 1. For example, in some embodiments, a tablet as described herein comprises 30 mg or 100 mg of Compound 1. In some embodiments, a tablet as described herein comprises 1 mg or 200 mg of Compound 1.

In some embodiments, methods described herein further comprise wherein the tablet is administered with food. In some embodiments, methods described herein further comprise wherein the tablet is administered with high-fat meal. In some embodiments, methods described herein further comprise wherein the tablet is administered with moderate-fat meal. In some embodiments, methods described herein further comprise wherein the tablet is administered with low-fat meal.

As used herein, the term “low-fat meal” or “light-fat meal” is a meal having about 400 kcal with about 20% of calories from fat.

As used herein, the term “moderate-fat meal” is a meal having about 600 kcal with about 27% of calories from fat.

As used herein, the term “high-fat meal” is a meal having about 800-1000 kcal with about 50% of calories from fat.

In some embodiments, methods described herein further comprise administering a therapeutically effective amount of selonsertib.

In some embodiments, methods described herein further comprise administering a therapeutically effective amount of firsocostat

Some embodiments provided herein are directed to a method of treating NASH in a patient in need thereof comprising administering a pharmaceutical composition comprising less than about 20% w/w, or 1% to 20% w/w, of a Compound 1, and at least one pharmaceutically acceptable carrier, and wherein

the pharmaceutical composition comprises about 30 mg, or 30 mg, of Compound 1; and

the percentage by weight is relative to the total weight of the pharmaceutical composition.

Some embodiments provided herein are directed to a method of treating NASH in a patient in need thereof comprising administering a pharmaceutical composition comprising less than about 25% w/w, or 1% to 25% w/w, of a Compound 1, or a pharmaceutically acceptable salt thereof, and at least one pharmaceutically acceptable carrier, and wherein

the pharmaceutical composition comprises about 30 mg, or 30 mg, of Compound 1; and

the percentage by weight is relative to the total weight of the pharmaceutical composition.

Some embodiments provided herein are directed to a method of treating NASH in a patient in need thereof comprising administering a pharmaceutical composition comprising about 12% w/w, or 12% w/w, of a Compound 1, and at least one pharmaceutically acceptable carrier, and wherein

the pharmaceutical composition comprises about 30 mg, or 30 mg, of Compound 1; and

the percentage by weight is relative to the total weight of the pharmaceutical composition.

Some embodiments provided herein are directed to a method of treating NASH in a patient in need thereof comprising administering a pharmaceutical composition comprising about 8% w/w, or 8% w/w, of a Compound 1, and at least one pharmaceutically acceptable carrier, and wherein

the pharmaceutical composition comprises about 30 mg, or 30 mg, of Compound 1; and

the percentage by weight is relative to the total weight of the pharmaceutical composition.

Some embodiments provided herein are directed to a method of treating PSC in a patient in need thereof comprising administering a pharmaceutical composition comprising less than about 20% w/w, or 1% to 20% w/w, of a Compound 1, and at least one pharmaceutically acceptable carrier, and wherein

the pharmaceutical composition comprises about 100 mg, or 100 mg, of Compound 1; and

the percentage by weight is relative to the total weight of the pharmaceutical composition.

Some embodiments provided herein are directed to a method of treating PSC in a patient in need thereof comprising administering a pharmaceutical composition comprising less than about 25% w/w, or 1% to 25% w/w, of a Compound 1, or a pharmaceutically acceptable salt thereof, and at least one pharmaceutically acceptable carrier, and wherein

the pharmaceutical composition comprises about 100 mg, or 100 mg, of Compound 1; and

the percentage by weight is relative to the total weight of the pharmaceutical composition.

Some embodiments provided herein are directed to a method of treating PSC in a patient in need thereof comprising administering a pharmaceutical composition comprising about 12% w/w, or 12% w/w, of a Compound 1, and at least one pharmaceutically acceptable carrier, and wherein

the pharmaceutical composition comprises about 100 mg, or 100 mg, of Compound 1; and

the percentage by weight is relative to the total weight of the pharmaceutical composition.

Some embodiments provided herein are directed to a method of treating PSC in a patient in need thereof comprising administering a pharmaceutical composition comprising about 8% w/w, or 8% w/w, of a Compound 1, and at least one pharmaceutically acceptable carrier, and wherein

the pharmaceutical composition comprises about 100 mg, or 100 mg, of Compound 1; and

the percentage by weight is relative to the total weight of the pharmaceutical composition.

Some embodiments provided herein are directed to a method of treating PSC in a patient in need thereof comprising administering a pharmaceutical composition comprising less than about 20% w/w, or 1% to 20% w/w, of a Compound 1, and at least one pharmaceutically acceptable carrier, and wherein

the pharmaceutical composition comprises about 30 mg, or 30 mg, of Compound 1; and

the percentage by weight is relative to the total weight of the pharmaceutical composition.

Some embodiments provided herein are directed to a method of treating PSC in a patient in need thereof comprising administering a pharmaceutical composition comprising less than about 25% w/w, or 1% to 25% w/w, of a Compound 1, or a pharmaceutically acceptable salt thereof, and at least one pharmaceutically acceptable carrier, and wherein

the pharmaceutical composition comprises about 30 mg, or 30 mg, of Compound 1; and

the percentage by weight is relative to the total weight of the pharmaceutical composition.

Some embodiments provided herein are directed to a method of treating PSC in a patient in need thereof comprising administering a pharmaceutical composition comprising about 12% w/w, or 12% w/w, of a Compound 1, and at least one pharmaceutically acceptable carrier, and wherein

the pharmaceutical composition comprises about 30 mg, or 30 mg, of Compound 1; and

the percentage by weight is relative to the total weight of the pharmaceutical composition.

Some embodiments provided herein are directed to a method of treating PSC in a patient in need thereof comprising administering a pharmaceutical composition comprising about 8% w/w, or 8% w/w, of a Compound 1, and at least one pharmaceutically acceptable carrier, and wherein

the pharmaceutical composition comprises about 30 mg, or 30 mg, of Compound 1; and

the percentage by weight is relative to the total weight of the pharmaceutical composition.

Medicaments as referred to herein may be prepared by conventional processes, including the combination of a compound according to the present disclosure and a pharmaceutically acceptable carrier.

### Kits

Provided herein are also kits that include a compound or composition described (e.g. such as a tablet described herein) herein and suitable packaging. In one embodiment, a kit further includes instructions for use. In one aspect, a kit includes a composition of the disclosure and a label and/or instructions for use of the compounds in the treatment of the indications, including the diseases or conditions, described herein.

Provided herein are also articles of manufacture that include a compound or composition described herein in a suitable container. The container may be a vial, jar, ampoule, preloaded syringe, and intravenous bag.

### Combination Therapy

In some embodiments, disclosed herein are oral dosage forms (e.g., tablets) comprising Compound 1:

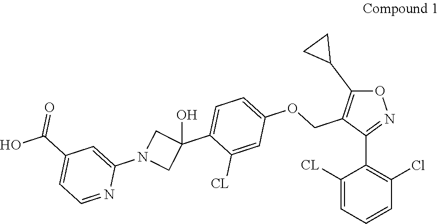

or a pharmaceutically acceptable salt thereof and at least one additional therapeutic agent. In some embodiments, the oral dosage forms disclosed herein comprise Compound 1 or a pharmaceutically acceptable salt thereof and one, two, or three additional therapeutic agents.

In some embodiments, the therapeutic agent, or combination of therapeutic agents, are a(n) ACE inhibitor, Acetaldehyde dehydrogenase inhibitor, Acetyl CoA carboxylase inhibitor, Acetyl CoA carboxylase inhibitor, Diacylglycerol O acyltransferase 2 inhibitor, Adenosine A3 receptor agonist, Adiponectin receptor agonist, Aldehyde dehydrogenase 2 stimulator, AKT protein kinase inhibitor, AMP-activated protein kinases (AMPK), AMP kinase activator, ATP citrate lyase inhibitor, AMP activated protein kinase stimulator, Endothelial nitric oxide synthase stimulator, NAD-dependent deacetylase sirtuin-1 stimulator, Androgen receptor agonist, Amylin receptor agonist, Angiotensin II AT-1 receptor antagonist, Autophagy protein modulator, Autotaxin inhibitors, Axl tyrosine kinase receptor inhibitor, Bax protein stimulator, Bioactive lipid, Calcitonin agonist, Cannabinoid receptor modulator, Caspase inhibitor, Caspase-3 stimulator, Cathepsin inhibitor, Caveolin 1 inhibitor, CCR2 chemokine antagonist, CCR2 chemokine antagonist, Angiotensin II AT-1 receptor antagonist, CCR3 chemokine antagonist, CCR5 chemokine antagonist, CD3 antagonist, Chloride channel stimulator, CNR1 inhibitor, Cyclin D1 inhibitor, Cytochrome P450 7A1 inhibitor, DGAT1/2 inhibitor, Diacylglycerol O acyltransferase 1 inhibitor (DGAT1), Cytochrome P450 2E1 inhibitor (CYP2E1), CXCR4 chemokine antagonist, Dipeptidyl peptidase IV inhibitor, Endosialin modulator, Eotaxin ligand inhibitor, Extracellular matrix protein modulator, Farnesoid X receptor agonist, Fatty acid synthase inhibitors, FGF1 receptor agonist, Fibroblast growth factor (FGF-15, FGF-19, FGF-21) ligands, Galectin-3 inhibitor, Glucagon receptor agonist, Glucagon-like peptide 1 agonist, G-protein coupled bile acid receptor 1 agonist, G-protein coupled receptor 84 antagonist, Hedgehog (Hh) modulator, Hepatitis C virus NS3 protease inhibitor, Hepatocyte nuclear factor 4 alpha modulator (HNF4A), Hepatocyte growth factor modulator, Histone deacetylase inhibitor, STAT-3 modulator, HMG CoA reductase inhibitor, Hypoxia inducible factor-2 alpha inhibitor, IL-10 agonist, IL-17 antagonist, Ileal sodium bile acid cotransporter inhibitor, Insulin sensitizer, Insulin ligand agonist, Insulin receptor agonist, integrin modulator, Integrin Antagonist, intereukin-1 receptor-associated kinase 4 (IRAK4) inhibitor, IL-6 receptor agonist, Jak2 tyrosine kinase inhibitor, Ketohexokinase (KHK) inhibitor, Klotho beta stimulator, 5-Lipoxygenase inhibitor, Lipoprotein lipase inhibitor, Liver X receptor, LPL gene stimulator, Lysophosphatidate-1 receptor antagonist, Lysyl oxidase homolog 2 inhibitor, Macrophage mannose receptor 1 modulator, Matrix metalloproteinases (MMPs) inhibitor, MEKK-5 protein kinase inhibitor, MCH receptor-1 antagonist, Membrane copper amine oxidase (VAP-1) inhibitor, Methionine aminopeptidase-2 inhibitor, Methyl CpG binding protein 2 modulator, MicroRNA-21(miR-21) inhibitor, Mitochondrial uncoupler, Mixed lineage kinase-3 inhibitor, Myelin basic protein stimulator, NACHT LRR PYD domain protein 3 (NLRP3) inhibitor , NAD-dependent deacetylase sirtuin stimulator, NADPH oxidase inhibitor (NOX), Nicotinic acid receptor 1 agonist, P2Y13 purinoceptor stimulator, Nuclear receptor modulators, P2X7 purinoceptor modulator, PDE 3 inhibitor, PDE 4 inhibitor, PDE 5 inhibitor, PDGF receptor beta modulator, Phenylalanine hydroxylase stimulator, Phospholipase C inhibitor, PPAR alpha agonist, PPAR delta agonist, PPAR gamma agonist, Peptidyl-prolyl cis-trans isomerase A inhibitor, PPAR gamma modulator, Protease-activated receptor-2 antagonist, Protein kinase modulator, Rho associated protein kinase inhibitor, Snitrosoglutathione reductase (GSNOR) enzyme inhibitor, Sodium glucose transporter-2 inhibitor, SREBP transcription factor inhibitor, STAT-1 inhibitor, Stearoyl CoA desaturase-1 inhibitor, STK25 inhibitor, Suppressor of cytokine signalling-1 stimulator, Suppressor of cytokine signalling-3 stimulator, Transforming growth factor β (TGF-β), Transforming growth factor β activated Kinase 1 (TAK1), Thyroid hormone receptor beta agonist, TLR-4 antagonist, Transglutaminase inhibitor, Tyrosine kinase receptor modulator, GPCR modulator, nuclear hormone receptor modulator, WNT modulators, or YAP/TAZ modulator and Zonulin inhibitor.

Non-limiting examples of the one or more additional therapeutic agents include:

ACE inhibitors, such as enalapril;

Acetaldehyde dehydrogenase inhibitors, such as ADX-629;

Acetyl CoA carboxylase (ACC) inhibitors, such as NDI-010976 (firsocostat), DRM-01, gemcabene, PF-05175157, QLT-091382, PF-0522 1304;

Acetyl CoA carboxylase/Diacylglycerol 0 acyltransferase 2 inhibitors, such as PF-07055341;

Adenosine receptor agonists, such as CF-102 (namodenoson), CF-101, CF-502, CGS21680;

Adiponectin receptor agonists, such as ADP-355, ADP-399;

Aldehyde dehydrogenase 2 stimulators, such as FP-045;

Amylin/calcitonin receptor agonists, such as KBP-042, KBP-089;

AMP activated protein kinase stimulators, such as, PXL-770, 0-304;

AMP kinase activators/ATP citrate lyase inhibitors, such as bempedoic acid (ETC-1002, ESP-55016)

AMP activated protein kinase/Endothelial nitric oxide synthase/NAD-dependent deacetylase sirtuin-1 stimulators, such as NS-0200;

Androgen receptor agonists, such as LPCN-1144;

Angiotensin II AT-1 receptor antagonists, such as irbesartan;

Angiopoietin-related protein-3 inhibitors, such as IONIS-ANGPTL3-LRx;

Autophagy protein modulators, such as A-2906;

Autotaxin inhibitors, such as PAT-505, PAT-048, GLPG-1690, X-165, PF-8380, AM-063, BBT-877;

Axl tyrosine kinase receptor inhibitors, such as bemcentinib (BGB-324, R-428);

Bax protein stimulators, such as CBL-514;

Bioactive lipids, such as DS-102;

Cannabinoid receptor modulators, such as namacizumab, GWP-42004, REV-200, CRB-4001;Caspase inhibitors, such as emricasan;

Pan cathepsin B inhibitors, such as VBY-376;

Pan cathepsin inhibitors, such as VBY-825;

CCR2/CCRS chemokine antagonists, such as cenicriviroc, maraviroc, CCX-872, WXSH-0213;

CCR2 chemokine antagonists, such as propagermanium;

CCR2 chemokine/Angiotensin II AT-1 receptor antagonists, such as DMX-200, DMX-250;

CCR3 chemokine antagonists, such as bertilimumab;

CD3 antagonists, such as NI-0401;

Chloride channel stimulators, such as cobiprostone;

CXCR4 chemokine antagonists, such as AD-214;

Diglyceride acyltransferase 2 (DGAT2) inhibitors, such as IONIS-DGAT2Rx, PF-06865571;

Diglyceride acyltransferase 1 (DGAT1) inhibitors, such as GSK-3008356;

Diacylglycerol O acyltransferase 1 (DGAT1)/Cytochrome P450 2E1 inhibitors (CYP2E1), such as SNP-610;

Dipeptidyl peptidase IV inhibitors, such as linagliptin, evogliptin;

Eotaxin ligand inhibitors, such as bertilimumab, CM-101;

Extracellular matrix protein modulators, such as CNX-024;

Farnesoid X receptor (FXR) agonists, such as AGN-242266, AGN-242256, EP-024297, RDX-023, BWL-200, AKN-083, EDP-305, GNF-5120, GS-9674, LMB-763, obeticholic acid, Px-102, Px-103, M790, M780, M450, M-480, (MET-409), PX20606, EYP-001, TERN-101, TC-100, INT-2228;

Farnesoid X receptor (FXR)/G-protein coupled bile acid receptor 1(TGRS) agonists, such as INT-767;

Fatty acid synthase inhibitors, such as TVB-2640;

Fibroblast growth factor 19 (rhFGF19)/cytochrome P450 (CYP) 7A1 inhibitors, such as NGM-282;

Fibroblast growth factor 21(FGF-21) ligand, such as BMS-986171, BI089-100, BMS-986036, B-1344;

Fibroblast growth factor 21(FGF-21)/glucagon like peptide 1 (GLP-1) agonist, such as YH-25723 AKR-001;

Galectin-3 inhibitors, such as GR-MD-02, GB-1107;

Glucagon-like peptide 1(GLP1R) agonists, such as AC-3174, liraglutide, cotadutide (MEDI-0382), SAR-425899, LY-3305677, HM-15211, YH-25723, YH-GLP1, RPC-8844, PB-718, semaglutide;

G-protein coupled bile acid receptor 1(TGRS) agonists, such as RDX-009, INT-777;

Heat shock protein 47 (HSP47) inhibitors, such as ND-L02-s0201;

Histone deacetylase inhibitors/STAT-3 modulators, such as SFX-01;

HMG CoA reductase inhibitors, such as atorvastatin, fluvastatin, pitavastatin, pravastatin, rosuvastatin, simvastatin;

Hypoxia inducible factor-2 alpha inhibitors, such as PT-2567;

IL-10 agonists, such as peg-ilodecakin;

Ileal sodium bile acid cotransporter inhibitors, such as odevixibat (A-4250), volixibat potassium ethanolate hydrate (SHP-262), GSK2330672, CJ-14199, elobixibat (A-3309);

Insulin sensitizers, such as, KBP-042, MSDC-0602K, MSDC-5514, Px-102, RG-125 (AZD4076), VVP-100X, CB-4211, ETI-101;

Insulin ligand/dslnsulin receptor agonists, such as ORMD-0801;

Integrin antagonists, such as IDL-2965;

IL-6 receptor agonists, such as KM-2702;

Ketohexokinase (KHK) inhibitors, such as PF-06835919;

beta Klotho (KLB)- FGF1c agonists, such as MK-3655 (NGM-313);

5-Lipoxygenase inhibitors, such as tipelukast (MN-001), DS-102 (AF-102);

Lipoprotein lipase inhibitors, such as CAT-2003;

LPL gene stimulators, such as alipogene tiparvovec;

Liver X receptor (LXR) inhibitors, such as PX-L603, PX-L493, BMS-852927, T-0901317, GW-3965, SR-9238;

Lysophosphatidate-1 receptor antagonists, such as BMT-053011, UD-009 (CP-2090), AR-479, ITMN-10534, BMS-986020, KI-16198;

Lysyl oxidase homolog 2 inhibitors, such as simtuzumab, PXS-5382A (PXS-5338);

Macrophage mannose receptor 1 modulators, such as tilmanocept-Cy3 (technetium Tc 99m tilmanocept);

Membrane copper amine oxidase (VAP-1) inhibitors, such as TERN-201;

MEKK-5 protein kinase (ASK-1) inhibitors, such as GS-4997, SRT-015, GS-444217, GST-HG-151;

MCH receptor-1 antagonists, such as CSTI-100 (ALB-127158);

Semicarbazide-Sensitive Amine Oxidase/Vascular Adhesion Protein-1 (SSAO/VAP-1) Inhibitors, such as PXS-4728A;

Methionine aminopeptidase-2 inhibitors, such as ZGN-1061, ZGN-839, ZN-1345;

Methyl CpG binding protein 2 modulators, such as mercaptamine;

Mineralocorticoid receptor antagonists (MCRA), such as MT-3995;

Mitochondrial uncouplers, such as 2,4-dinitrophenol;

Mixed lineage kinase-3 inhibitors, such as URMC-099-C;

Myelin basic protein stimulators, such as olesoxime;

Myeloperoxidase inhibitors, such as PF-06667272, AZM-198;

NADPH oxidase inhibitors, such as GKT-831, APX-311; Nicotinic acid receptor 1 agonists, such as ARI-3037M0;

NACHT LRR PYD domain protein 3 (NLRP3) inhibitors, such as KDDF-201406-03, NBC-6, IFM-514, JT-194 (JT-349);

Nuclear receptor modulators, such as DUR-928 (DV-928);

P2X7 purinoceptor modulators, such as SGM-1019;

P2Y13 purinoceptor stimulators, such as CER-209;

PDE 3/4 inhibitors, such as tipelukast (MN-001);

PDE 5 inhibitors, such as sildenafil, MSTM-102;

PDGF receptor beta modulators, such as BOT-191, BOT-509;

Peptidyl-prolyl cis-trans isomerase inhibitors, such as CRV-431 (CPI-432-32), NVP-018, NV-556 (NVP-025);

Phenylalanine hydroxylase stimulators, such as HepaStem;

PPAR agonists, such as elafibranor (GFT-505), seladelpar lysine (MBX-8025), deuterated pioglitazone R-enantiomer, pioglitazone, DRX-065, saroglitazar, lanifibranor (IVA-337), CHS-131;

Protease-activated receptor-2 antagonists, such as PZ-235;

Protein kinase modulators, such as CNX-014;

Rho associated protein kinase (ROCK) inhibitors, such as REDX-10178 (REDX-10325), KD-025;

Snitrosoglutathione reductase (GSNOR) enzyme inhibitors, such as SL-891;

Sodium glucose transporter-2(SGLT2) inhibitors, such as ipragliflozin, remogliflozin etabonate, ertugliflozin, dapagliflozin, tofogliflozin, sotagliflozin,

Sodium glucose transporter-1/2 (SGLT 1/2) inhibitors, such as licogliflozin bis(prolinate);

SREBP transcription factor inhibitors, such as CAT-2003, MDV-4463;

Stearoyl CoA desaturase-1 inhibitors, such as aramchol;

Thyroid hormone receptor beta agonists, such as resmetirom (MGL-3196), MGL-3745, VK-2809;

TLR-2/TLR-4 antagonists, such as VB-201 (CI-201);

TLR-4 antagonists, such as JKB-121;

Tyrosine kinase receptor modulators, such as CNX-025;

GPCR modulators, such as CNX-023;

Nuclear hormone receptor modulators, such as Px-102;

Xanthine oxidase/Urate anion exchanger 1 (URAT1) inhibitors, such as RLBN-1001, RLBN-1127; and

Zonulin Inhibitors, such as lorazotide acetate (INN-202).

In certain specific embodiments, the one or more additional therapeutic agents are selected from A-4250, AC-3174, acetylsalicylic acid, AK-20, alipogene tiparvovec, AMX-342, AN-3015, aramchol, ARI-3037M0, ASP-8232, AZD-2693, bertilimumab, Betaine anhydrous, BI-1467335, BMS-986036, BMS-986171, BMT-053011, BOT-191, BTT-1023, CAT-2003, cenicriviroc, CBW-511, CER-209, CF-102, CGS21680, CNX-014, CNX-023, CNX-024, CNX-025, cobiprostone, colesevelam, dapagliflozin, DCR-LIV1, deuterated pioglitazone R-enantiomer, 2,4-dinitrophenol, DRX-065, DS-102, DUR-928, EDP-305, elafibranor (GFT-505), emricasan, enalapril, ertugliflozin, evogliptin, F-351, fluasterone (ST-002), FT-4101, GKT-831, GNF-5120, GRI-0621, GR-MD-02, GS-300, GS-4997, GS-9674, HTD-1801, HST-202, HST-201, hydrochlorothiazide, icosabutate (PRC-4016), icosapent ethyl ester, IMM-124-E, INT-767, INV-240, IONIS-DGAT2Rx, ipragliflozin, Irbesarta, propagermanium, IVA-337, JKB-121, KB-GE-001, KBP-042, KD-025, M790, M780, M450, metformin, sildenafil, LC-280126, linagliptin, liraglutide, LJN-452, LM-011, LM-002 (CVI-LM-002), LMB-763, LYN-100, MBX-8025, MDV-4463, mercaptamine, MGL-3196, MGL-3745, MP-301, MSDC-0602K, namacizumab, NC-101, NDI-010976, ND-L02-s0201, NGM-282, NGM-313, NGM-386, NGM-395, NP-160, norursodeoxycholic acid, NVP-022, 0-304, obeticholic acid, 25HC3S, olesoxime, PAT-505, PAT-048, PB-4547, peg-ilodecakin, pioglitazone, pirfenidone, PRI-724, PX20606, Px-102, PX-L603, PX-L493, PXS-4728A, PZ-235, RDX-009, remogliflozin etabonate, RG-125 (AZD4076), RPI-500, saroglitazar, semaglutide, simtuzumab, solithromycin, sotagliflozin, statins (atorvastatin, fluvastatin, pitavastatin, pravastatin, rosuvastatin, simvastatin), TCM-606F, TEV-45478, TQA-3526, tipelukast (MN-001), TLY-012, TRX-318, TVB-2640, UD-009, ursodeoxycholic acid, VBY-376, VBY-825, VK-2809, vismodegib, volixibat potassium ethanolate hydrate (SHP-626), VVP-100X, WAV-301, WNT-974, XRx-117, ZGN-839, ZG-5216, ZSYM-008, ZYSM-007.

In some embodiments, methods and compositions include a therapeutically effective amount of an Apoptosis Signal-Regulating Kinase 1 (ASK1) inhibitor and a therapeutically effective amount of a Farnesoid X Receptor (FXR) agonist, wherein the FXR agonist is a compound described herein.

In certain embodiments of the methods and pharmaceutical compositions disclosed herein, the ASK1 inhibitor is a compound of Formula (II):

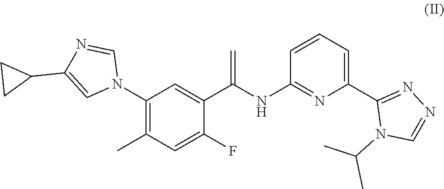 [SMILES: C=C(Nc1cccc(-c2nncn2C(C)C)n1)c1cc(-n2cnc(C3CC3)c2)c(C)cc1F]

or a pharmaceutically acceptable salt, a stereoisomer, a mixture of stereoisomers, or a tautomer thereof. A compound of Formula (II) is also known as GS-4997 or selonsertib.

ASK1 inhibitors, such as the compound of Formula (II), can be synthesized and characterized using methods known to those of skill in the art, such as those described in U.S. Pat. application publication No. 2007/0276050, U.S. Patent Application Publication No. 2011/0009410, and U.S. Patent Application Publication No. 2013/0197037.

In some embodiments, methods and compositions include a therapeutically effective amount of an Acetyl CoA Carboxylase inhibitor and a therapeutically effective amount of a Farnesoid X Receptor (FXR) agonist, wherein the FXR agonist is a solid form described herein.

In certain embodiments of the methods and pharmaceutical compositions disclosed herein, the ACC inhibitor is a compound of Formula (III):

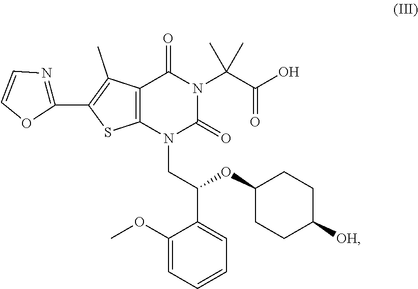 [SMILES: COc1ccccc1[C@H](Cn1c(=O)n(C(C)(C)C(=O)O)c(=O)c2c(C)c(-c3ncco3)sc21)O[C@H]1CC[C@@H](O)CC1]

or a pharmaceutically acceptable salt thereof.

A compound of Formula (III) is also known as GS-0976 or NDI-010976 or firsocostat.

In certain embodiments of the methods and pharmaceutical compositions disclosed herein, the ACC inhibitor is a compound having the structure of Formula (IV):

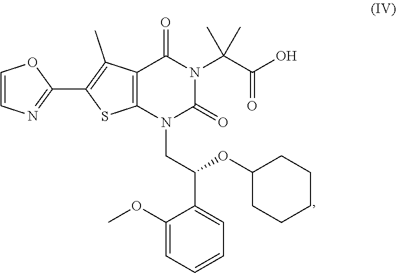 [SMILES: COc1ccccc1[C@H](Cn1c(=O)n(C(C)(C)C(=O)O)c(=O)c2c(C)c(-c3ncco3)sc21)OC1CCCCC1]

or a pharmaceutically acceptable salt thereof.

The compounds of Formula (III) and Formula (IV) may be synthesized and characterized using methods known to those of skill in the art, such as those described in International Application Publication No. WO 2013/071169.

In certain embodiments of the methods and pharmaceutical compositions disclosed herein, the ASK1 inhibitor is a compound of Formula (II), the ACC inhibitor is a compound of Formula (III), and the FXR agonist is a compound of Formula (I).

### EXAMPLES

### Preparation of Compound 1

Compound 1 is synthesized according to known methods, such as those disclosed in U.S. Pat. No. 9,139,539. Formula I Tromethamine Salt (Form I) for use in the tablets described herein can be prepared as follows.

Compound 1 tromethamine salt (tris salt) Form I was obtained by drying Compound 1 tromethamine salt ethanol solvate (at 0% RH and 25° C.). Compound 1 tromethamine salt ethanol solvate was obtained by charging a 4 mL vial with 52.5 mg of zwitterionic Compound 1:

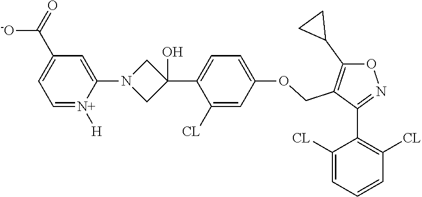

about 1.1 equivalent Tris (12 mg) and 1 mL of ethanol. The slurry was stirred at about 50° C. for about 5 hours and at room temperature overnight. The sample of wet solids was analyzed by XRPD and afforded unique XRPD pattern of ethanol solvate, which converted to Compound 1 tromethamine salt hydrate I at ambient conditions. Alternative methods for providing Compound 1 tromethamine salt (tris salt), including, but not limited to, Form I, for use in the tablets described herein, can be found in U.S. Patent application Ser. No. 16/791,974, filed Feb. 14, 2020, which is hereby incorporated by reference in its entirety, with pages 62-80 of the specification, as filed, incorporated hereby with specificity.

XRPD patterns were collected on a PANanalytical XPERT-PRO diffractometer at ambient conditions under the following experimental settings: 45 KV, 40 mA, Kα1=1.5406 Å, scan range 2 to 40° 2θ, step size 0.0084 or 0.0167° 2θ, measurement time: 5 min. XRPD analysis of Compound 1 tromethamine salt Form I shows an XRPD pattern comprising degree 2θ-reflections (±-0.2 degrees 2θ) at 5.2, 16.8, and 25.6 degrees. In some embodiments, Formula I tromethamine salt Form I has an XRPD pattern comprising degree 2θ-reflections (±0.2 degrees 2θ) at 5.2, 16.8, and 25.6 degrees and one, two, or three of the degree 2θ-reflections (±0.2 degrees 2θ) at 10.9, 15.3, and 21.8 degrees. In some embodiments, Formula I tromethamine salt Form I has an XRPD pattern comprising degree 2θ-reflections (±0.2 degrees 2θ) at 5.2, 16.8, and 25.6 degrees and one, two, or three of the degree 2θ-reflections (±0.2 degrees 2θ) at 10.9, 15.3, and 21.8 degrees. In some embodiments, Formula I tromethamine salt Form I has an XRPD pattern comprising degree 2θ-reflections (±0.2 degrees 20) at 5.2, 16.8, and 25.6 degrees and one, two, three, four, or five of the degree 2θ-reflections (±0.2 degrees 20) at 13.3, 20.1, 20.4, 21.0, and 24.3 degrees. In some embodiments, Formula I tromethamine salt Form I has an XRPD pattern comprising degree 2θ-reflections (±0.2 degrees 2θ) at 5.2, 16.8, 25.6, 10.9, 15.3, 21.8, and 13.3, 20.1, 20.4, 21.0, and 24.3.

DSC analysis of Compound 1 tromethamine salt Form I shows a melting onset at about 129° C., followed by exotherm with onset at about 150° C. and decomposition.

TGA analysis of Compound 1 tromethamine salt Form I shows the solids did not show any weight loss below about 150° C. prior to decomposition.

### Example 1: Tablet Preparation and Formulation

Exemplary powder formulations of Compound 1 are shown in Table 1, Table 2, and Table 3 below. These formulations were prepared as follows. A tromethamine salt of Compound 1 was blended with microcrystalline cellulose, mannitol and crospovidone. The blend was passed through a mill and then blended with the intragranular portion of magnesium stearate. The powder blend was roller compacted and passed through a mill. The resulting granules were blended with the extragranular portion of magnesium stearate and compressed into core tablets and film-coated.

TABLE 1

| | Formulation 1 | | Formulation 2 | | Formulation 3 | |
|---|---|---|---|---|---|---|
| | | Component | | Component | | Component |
| | Amount | Weight | Amount | Weight | Amount | Weight |
| Components | (% w/w) | (mg/tablet) | (% w/w) | (mg/tablet) | (% w/w) | (mg/tablet) |
| Tromethamine Salt of | 6.03^{a} | 12.06 | 24.12^{a} | 120.60 | 1.21^{a} | 1.21 |
| Compound 1 | | | | | | |
| Microcrystalline | 57.50 | 115.00 | 45.00 | 225.00 | 62.00 | 62.00 |
| Cellulose | | | | | | |
| Lactose | 27.97 | 55.94 | 22.38 | 111.90 | 30.79 | 30.79 |
| Monohydrate | | | | | | |
| Crospovidone | 7.00 | 14.00 | 7.00 | 35.00 | 5.00 | 5.00 |
| Magnesium | 1.50^{b} | 3.00 | 1.50^{b} | 7.50 | 1.00^{c} | 1.00 |
| Stearate | | | | | | |
| Total | 100 | 200 | 100 | 500 | 100 | 100 |
| Film-Coating | | | | | | |
| Opadry II Orange | 3 | 6 | 3 | 15 | 3 | 3 |
| 85F93558 | | | | | | |
| Purified Water^{d} | N/A | N/A | N/A | N/A | N/A | N/A |
| Total Film-Coated | | 206 | | 515 | | 103 |
| Tablet Weight | | | | | | |

^{a}6.03% (w/w) was the amount of a tromethamine salt of Compound 1 used to generate a composition containing 5.00% (w/w) of Compound 1 (zwitterion). 24.12% (w/w) was the amount of Tromethamine Salt of Compound 1 used to generate a composition containing 20.00% (w/w) of Compound 1 (zwitterion). 1.21% (w/w) was the amount of a tromethamine salt of Compound 1 used to generate a composition containing 1.00% (w/w) of Compound 1 (zwitterion).
^{b}0.75% intragranular; 0.75% extragranular.
^{c}0.50% intragranular; 0.50% extragranular.
^{d}Purified water is used and removed during the film-coating process.

TABLE 2

| | Formulation 4 | | Formulation 5 | | | |
|---|---|---|---|---|---|---|
| | | Component | | Component | Formulation 6 | |
| | Amount | Weight | Amount | Weight | Amount | Component Weight |
| Components | (% w/w) | (mg/tablet) | (% w/w) | (mg/tablet) | (% w/w) | (mg/tablet) |
| Tromethamine Salt of | 24.12^{a} | 36.18 | 24.12^{a} | 120.60 | 6.03^{a} | 36.18 |
| Compound 1 | | | | | | |
| Microcrystalline | 45.00 | 67.50 | 45.00 | 225.00 | 28.40 | 170.40 |
| Cellulose | | | | | | |
| Mannitol | 22.38 | 33.57 | 22.38 | 111.90 | 56.82 | 340.92 |
| Crospovidone | 7.00 | 10.50 | 7.00 | 35.00 | 7.00 | 42.00 |
| Magnesium | 1.50^{b} | 2.25 | 1.50^{b} | 7.50 | 1.75^{c} | 10.50 |
| Stearate | | | | | | |
| Total | 100 | 150 | 100 | 500 | 100 | 600 |
| Film-Coating | | | | | | |
| Opadry II Orange | 3 | 4.5 | 3 | 15 | 3 | 18 |
| 85F93558 | | | | | | |
| Purified Water^{d} | N/A | N/A | N/A | N/A | N/A | N/A |
| Total Film-Coated | | 155 | | 515 | | 618 |
| Tablet Weight | | | | | | |

^{a}24.12% (w/w) was the amount of a tromethamine salt of Compound 1 used to generate a composition containing 20.00% (w/w) of Compound 1 (zwitterion). 6.03% (w/w) was the amount of a tromethamine salt of Compound 1 used to generate a composition containing 5.00% (w/w) of Compound 1 (zwitterion).
^{b}0.75% intragranular; 0.75% extragranular.
^{c}0.75% intragranular; 1.00% extragranular.
^{d}Purified water is used and removed during the film-coating process.

TABLE 3

| | Formulation 7 | | Formulation 8 | | Formulation 9 | |
|---|---|---|---|---|---|---|
| | | Component | | Component | | Component |
| | Amount | Weight | Amount | Weight | Amount | Weight |
| Components | (% w/w) | (mg/tablet) | (% w/w) | (mg/tablet) | (% w/w) | (mg/tablet) |
| Tromethamine Salt of | 9.65^{a} | 120.63 | 14.361^{a} | 36.19 | 14.36^{a} | 120.63 |
| Compound 1 | | | | | | |
| Crospovidone | 7.00 | 87.50 | 7.00 | 17.64 | 7.00 | 58.80 |
| Mannitol | 54.60 | 682.50 | 51.39 | 129.50 | 51.39 | 431.67 |
| Microcrystalline | 27.00 | 337.50 | 25.50 | 64.26 | 25.50 | 214.20 |
| Cellulose | | | | | | |
| Magnesium | 1.75^{b} | 21.88 | 1.75^{b} | 4.41 | 1.75^{b} | 14.70 |
| Stearate | | | | | | |
| Total | 100 | 1250 | 100 | 252 | 100 | 840 |
| Film-Coating | | | | | | |
| Opadry II Green | 3 | 37.50 | 3.00 | 7.56 | 3.00 | 25.2 |
| 85F91177 | | | | | | |
| Purified Water^{c} | N/A | N/A | N/A | N/A | N/A | N/A |
| Total Film-Coated | | 1288 | | 260 | | 865 |
| Tablet Weight | | | | | | |

^{a}9.65% (w/w) was the amount of a tromethamine salt of Compound 1 used to generate a composition containing 8.00% (w/w) of Compound 1 (zwitterion). 14.36% (w/w) was the amount of a tromethamine salt of Compound 1 used to generate a composition containing 11.91% (w/w) of Compound 1 (zwitterion).
^{b}0.75% intragranular; 1.00% extragranular
^{c}Purified water is used and removed during the film-coating process.

### Example 2: Study Protocols

Study protocols as discussed in Examples 3 and 4 are as follows.

### Study A

Cohorts of Study A:

Part A: prespecified cohorts (Cohorts 1-3): Randomized, partially blind, placebo-controlled, single- and multiple-doses with staggered dose-escalations. 60 unique subjects; 15 per cohort (12 Compound 1, 3 placebo-to-match (“PTM”))

Part B: adaptive cohorts (Cohorts 5 and 8): Randomized, partially blind, placebo-controlled, single- and multiple-doses with adaptive dose selection and dose frequency. 60 unique subjects 15 per cohort (12 Compound 1, 3 PTM)

Target Population: Healthy male and non-pregnant, non-lactating female subjects 18-45 years of age, inclusive.

Eligible subjects within each cohort were an approximately even distribution of healthy male and non-pregnant, non-lactating female volunteers, with a body mass index (“BMI”) 19≤BMI≤30 kg/m², normal 12-lead electrocardiogram (“ECG”) or one with abnormalities that were considered clinically insignificant by the investigator, normal renal function (estimated glomerular filtration rate calculated using the Cockcroft-Gault equation≥80 mL/min), no significant medical history, and in good general health as determined by the investigator at Screening evaluation performed no more than 28 days prior to the scheduled first dose.

### Study Procedures/Frequency:

Part A (Single- and Multiple-Ascending Doses, Pre-Specified Cohorts) proceeded in 4 staggered, pre-specified, dose-escalation cohorts and were governed by study specific stopping criteria. Within each cohort, 15 unique subjects were randomized 4:1 to receive blinded Compound 1 (N=12) or PTM (N=3). All study drugs in Part A were administered in a fasted state. Within each cohort, dose escalation from single-dose (Period 1) to multiple-dose (Period 2) were permitted after evaluation of cumulative blinded safety data up to and through Day 3 of the same cohort.

The cohorts and study treatments for Part A are shown in Table 4:

TABLE 4

| | Baseline | Period 1 | Period 2 |
|---|---|---|---|
| Cohort | Day −1 | Day 1 | Day 7-20 |
| 1 | placebo | 10 mg Compound 1 | 10 mg Compound 1 |
| | tablet | (1 × 10 mg) or | (1 × 10 mg) or |
| | single dose | placebo tablet SD | (1 × 10 mg) or |
| | (“SD”) | | placebo tablet daily |
| 2 | placebo | 30 mg Compound 1 | 30 mg Compound 1 |
| | tablets SD | (1 × 10 mg) or | (1 × 10 mg) or |
| | | (3 × 10 mg) or | (3 × 10 mg) or |
| | | placebo tablets SD | placebo tablets daily |
| 3 | placebo | 100 mg Compound 1 | 100 mg Compound 1 |
| | tablet SD | (1 × 10 mg) or | (1 × 10 mg) or |
| | | (1 × 100 mg) or | (1 × 100 mg) or |
| | | placebo tablet SD | placebo tablet daily |

Part B (Adaptive Cohorts) is as follows: Based on available safety, pharmacokinetic (“PK”), and/or pharmacodynamic (“PD”) data from Part A, total daily doses for Part B (Cohorts 5 and 8) were chosen between 1 and 600 mg as well as frequency of dosing (once daily or twice a day) and meal conditions for dosing (fasted, low fat, moderate fat, or high fat meal). Once determined, dose level, frequency of dosing, and meal conditions were consistent within a cohort.

If doses chosen in 2 or more adaptive cohorts exceeded the dose evaluated in a previous cohort, those cohorts were conducted in a staggered manner (lowest dose first) similar to cohorts in Part A, with the same stopping rules applied. Part B cohorts were potentially initiated in parallel with cohorts in Part A if the total dose under evaluation was at or below a dose already evaluated.

Within each cohort, 15 unique subjects were randomized 4:1 to receive up to 600 mg Compound 1 (N=12) or PTM (N=3). If Compound 1/PTM was administered twice a day, the total daily dose did not exceed 600 mg. Study treatments within each cohort were administered either once daily or twice a day in either the fasted or fed (low, moderate, or high fat meal) state.

The cohorts and study treatments for Part B are as shown in Table 5:

TABLE 5

| | Baseline | Period 1 | Period 2 |
|---|---|---|---|
| Cohorts | Day −1 | Day 1 | Day 7-20 |
| 5 and 8 | Placebo | up to 600 mg | up to 600 mg |
| | tablet(s) | Compound 1 or | Compound 1 or |
| | SD | placebo tablet(s) | placebo tablet(s) |
| | | once daily or | once daily or |
| | | twice a day | twice a day |

The study drug(s) were supplied as Compound 1 tablets, in strengths of 1 mg, 10 mg, and 100 mg. Placebo-to-match Compound 1 tablets that did not contain Compound 1 were also be supplied and were identical in size, shape, color and appearance to their corresponding strengths of active Compound 1 tablets.

All study treatments were administered with 240 mL of water. For study treatments including more than 8 tablets, up to an additional 60 mL of water was administered if necessary.

All study treatments in Part A were administered in the fasted state as described below. Study Treatments in Part B were administered in the fasted or fed state as described below.

Fasted State Dosing: Study drug(s) were administered at approximately the same time each day following an overnight fast (no food or drinks except water, for at least 10 hours). Subjects continued to fast until after collection of the 2-hour PK sample, relative to study drug dosing.

On days of intensive PK and/or PD sampling, all study drug(s) were administered at approximately the same time each day following an overnight fast (no food or drinks except water, for at least 10 hours). Subjects continued to fast until after collection of the 4-hour PK sample, relative to study drug dosing. Additionally, subjects were restricted from water consumption from 1 hour before through hours after dosing, except for the water given with the study drug(s). A standardized meal may be provided to subjects after the 4-hour post-dose PK draw.

Fed State Dosing: Study drug(s) were administered at approximately the same time each day and within 5 minutes of completing a standardized meal. The meal was initiated 30 minutes prior to study drug administration. Subjects fasted until after collection of the 4-hour PK sample, relative to study drug dosing. Meal fat content (low fat, moderate fat, or high fat) was determined based on available data from subsequent cohorts.

On days of intensive PK and/or PD sampling, all study drug(s) were administered at approximately the same time each day following an overnight fast (no food or drinks except water, for at least 10 hours) and within 5 minutes of completing a standardized meal for dosing in the fed state. The meal should be initiated 30 minutes prior to study drug administration. Subjects continued to fast until after collection of the 4-hour PK sample, relative to study drug dosing. Additionally, subjects were restricted from water consumption from 1 hour before through 2 hours after dosing, except for the water given with the study drug(s) and the standardized meal. A standardized meal may be provided to subjects after the 4-hour post-dose PK draw.

### Study B

Cohorts of Study B, Part A (Relative bioavailability (“rBA”)):
Cohort 1: 20 subjects total (for 18 evaluable)
Cohort 3: 30 subjects total (for 26 evaluable)

Target Population: Healthy male and nonpregnant, nonlactating female subjects, aged 18-45, inclusive.

Eligible subjects were an approximately even distribution of healthy male and nonpregnant, nonlactating female subjects, with a body mass index (BMI)≥19.0 and ≤30.0 kg/m², normal 12-lead ECG, normal renal function, no significant medical history, and in good general health as determined by the Investigator at Screening evaluation performed no more than 28 days prior to the scheduled first dose.

Study Procedures/Frequency:

For Part A, once eligibility has been confirmed following completion of the admission (Day−2) study procedures, eligible subjects were randomized 1:1 to one of two treatment sequences within their respective cohort and assigned a subject number on Day−1 to receive study drug starting on Day 1.

Study Treatments are as follows:

Cohort 1 (selonsertib (“SEL”) and Compound 1):

Treatment A: Single dose of SEL 18 mg (1×18 mg tablet)+Compound 1 30 mg (1×30 mg tablet) coadministered orally in the morning within 5 minutes of completing a high-fat meal Treatment C: Single dose of Compound 1 30 mg (1×30 mg tablet) administered orally in the morning in the fasted state

Cohort 3 (Compound 1):

Treatment I: Single dose of Compound 1 30 mg (1×30 mg tablet) administered orally in the morning within 5 minutes of completing a light meal

On Day1 −1, the timing of meals and meal types were matched to Day 1, with the exception of Cohort 3 which were matched to Day 17.

Fasted Administration (Treatment C): Study drug(s) were administered in the morning following an overnight fast (no food or drinks except water for at least 10 hours). Subjects continued to fast until after collection of the 4-hour PK sample, relative to (first) study drug dosing. Additionally, subjects were restricted from water consumption 1 hour before until 2 hours after each study drug dosing, except for the 240 mL given with each study drug administration. Water may be consumed by subjects following the 2-hour blood draw for the remainder of the collection period. A meal (standardized lunch) was provided to subjects after the 4 hour postdose blood draw.

Light Meal Administration (Treatment I): Study drug(s) were administered with food and with 240 mL of water. Following an overnight fast (no food or drinks except water for at least 10 hours), a meal was initiated 30 minutes prior to study drug administration. The dose was administered at or within 5 minutes of the subjects completing (100%) of the provided light meal containing ˜400 kcal with ˜20% of the calories from fat. Subjects fasted for 4 hours after study drug administration. A meal (standardized lunch) was provided to subjects after the 4-hour post-dose blood draw. Additionally, water and other fluids was withheld 1 hour before until 2-hours after dose administration other than the water provided with dosing and beverages provided with the standardized meal (where applicable). Water may be consumed by subjects following the 2-hour blood draw for the remainder of the collection period.

High-Fat Meal Administration (Treatment A): Study drug(s) were administered with food and with 240 mL of water. Following an overnight fast (no food or drinks except water for at least 10 hours), a meal was initiated 30 minutes prior to study drug administration. The dose was administered at or within 5 minutes of the subjects completing (100%) of the provided high-fat meal containing ˜800-1000 kcal with -50% of calories from fat (approximately 150, 250, and 500-600 kcal from protein, carbohydrate, and fat, respectively). Subjects fasted for 4 hours after study drug administration. A meal (standardized lunch) was provided to subjects after the 4-hour post-dose blood draw. Additionally, water and other fluids was withheld 1 hour before until 2 hours after dose administration other than the water provided with dosing and beverages provided with the standardized meal (where applicable). Water may be consumed by subjects following the 2-hour blood draw for the remainder of the collection period.

### Study C

Total number of subjects planned: approximately 40 subjects in total (20 of which were Caucasian).

Eligible Caucasian subjects were an approximately even distribution of healthy males and non-pregnant, non-lactating female volunteers between 18-55 years, inclusive, with a BMI between 18 and 30 kg/m² (inclusive), nonsmoking, must have either a normal 12-lead ECG or one with abnormalities that are considered clinically insignificant by the Investigator, normal renal function (Clcr≥90 m1/min), no significant medical history, and be in good general health as determined at Screening evaluation performed no more than 28 days prior to the scheduled dose of study medication. Caucasian subjects were not of Japanese or Asian or African descent. Caucasian subjects' parents and grandparents were not of Japanese or Asian or African descent.

Eligible subjects received the following treatment: Single dose of 100 mg Compound 1 (1×100 mg tablet), administered orally in the morning on Day 1 following an overnight fast.

Each dose of study drug was administered in the morning on Day 1 with 240 mL of still (non-carbonated) water following an overnight fast (no food or drinks except water, for at least 10 hours). Subjects continued to fast and were restricted from food intake until after collection of the 4-hour blood draw. Additionally, subjects were restricted from consumption of water or other fluids 1 hour before until 2 hours after dosing, except for the 240 mL given with the study treatment.

### Study D

Cohorts of Study D are as follows:

Cohort 10 (Compound 1 100 mg, formulation 9 in Table 3):

Treatment D: Single dose of Compound 1 (1×100 mg tablet) administered orally in the morning within 5 minutes of completing a light-fat meal.

Treatment E: Single dose of Compound 1 (1×100 mg tablet) administered orally in the morning within 5 minutes of completing a high-fat meal.

Treatment F: Single dose of Compound 1 (1×100 mg tablet) administered orally in the morning in the fasted state.

TABLE 9
Summary of Compound 1 Exposure, Variability,
and Change in Exposure with Meal-Type

| Treatment | Study Day | | | | |
|---|---|---|---|---|---|
| Sequence | 1 | 2-8 | 9 | 10-16 | 17 |
| DEF | Compound 1 | WO | Compound 1 | WO | Compound 1 |
| (N = 10) | (light-fat | | (high-fat | | (fasted) |
| | meal) | | meal) | | |
| FDE | Compound 1 | WO | Compound 1 | WO | Compound 1 |
| (N = 10) | (fasted) | | (light-fat | | (high-fat |
| | | | meal) | | meal) |

WO = washout

### Fasting and Meals

All meals and/or snacks given to subjects during their stay in the clinical study facility were standardized for all subjects and were similar in calorie and fat content and taken at approximately the same time each day. Components of meals (e.g., margarine, jelly, bread) are given to subjects in individual portions (e.g., 1 tablespoon) per the approved meal schedule. The provision of meal components in bulk (e.g., a jar of jelly for subjects to share) are not be practiced. All meals were given at approximately the same time each day (e.g., 07:30, 12:00, and 18:00).

When study drug administration and intensive PK sampling occurred at the same timepoint, the PK sample was collected at the nominal timepoint and study drug administration occurred after the PK sample collection (within 5 minutes of the nominal timepoint).

### Fasted Administration: Treatments F

Compound 1 was administered in the morning following an overnight fast (no food or drinks except water for at least 10 hours). Subjects continued to fast until after collection of the 4-hour PK sample, relative to the (first) study drug dosing. Additionally, subjects were restricted from water consumption 1 hour before until 2 hours after each study drug dosing, except for the 240 mL given with each study drug administration. Water was optionally consumed by subjects following the 2-hour blood draw for the remainder of the collection period. A meal (standardized lunch) is provided to subjects after the 4-hour post-dose blood draw.

### Fed (Light Meal) Administration: Treatment D

Compound 1 was administered with food and with 240 mL of water. Following an overnight fast (no food or drinks except water for at least 10 hours), a meal was initiated 30 minutes prior to study drug administration. The dose was administered at or within 5 minutes of the subjects completing (100%) of the provided light meal containing ˜400 kcal with ˜20% of the calories from fat. Subjects fasted for 4 hours after study drug administration. A meal (standardized lunch) is provided to subjects after the 4-hour post-dose blood draw.

Additionally, water and other fluids were withheld 1 hour before until 2 hours after dose administration other than the water provided with dosing and beverages provided with the standardized meal (where applicable). Water was optionally consumed by subjects following the 2-hour blood draw for the remainder of the collection period.

### Fed (High-Fat Meal) Administration: Treatment E

Compound 1 was administered with food and with 240 mL of water. Following an overnight fast (no food or drinks except water for at least 10 hours), a meal was initiated 30 minutes prior to study drug administration. The dose was administered at or within 5 minutes of the subjects completing (100%) of the provided high-fat meal containing ˜800-1,000 kcal with ˜50% of calories from fat (approximately 150, 250, and 500-600 kcal from protein, carbohydrate, and fat, respectively). Subjects fasted for 4 hours after study drug administration. A meal (standardized lunch) will be provided to subjects after the 4 hour postdose blood draw.

Additionally, water and other fluids were withheld 1 hour before until 2 hours after dose administration other than the water provided with dosing and beverages provided with the standardized meal (where applicable). Water was optionally consumed by subjects following the 2-hour blood draw for the remainder of the collection period.

### Example 3: Effect of Drug Load on Variability of Compound 1 Exposure

Single dose pharmacokinetic exposure parameters (AUC_{inf}) of Compound 1 from multiple Phase 1 studies in healthy volunteers which utilized various drug loads of Compound 1 were compared to determine if Compound 1 drug load impacted the variability and/or absolute value of Compound 1 systemic exposure. Data used in this analysis are presented in Table 6 below.

TABLE 6
Description of Data Used in this Analysis

| | | | | | Study #, |
|---|---|---|---|---|---|
| Compound 1 | Meal | Compound 1 | N | | Cohort/Treatment |
| Dose (mg) | Type | Drug Load (%) | (Subjects) | Dosage Form | or Population |
| 30 mg | Fasted | 20% | 20 | Compound 1 | Study B, 1/C |
| | | | | 30 mg Tablet | |
| | | 5% | 12 | Compound 1 | Study A, 2 |
| | | | | 10 mg Tablet | |
| 100 mg | Fasted | 20% | 12 | Compound 1 | Study A, 3 |
| | | | | 100 mg Tablet | |
| | | 8% | 20 | Compound 1 | Study C, |
| | | | | 100 mg Tablet | Caucasian Subjects |
| | | 12% | 20 | Compound 1 | Study D, 10/F |
| | | | | 100 mg Tablet | |

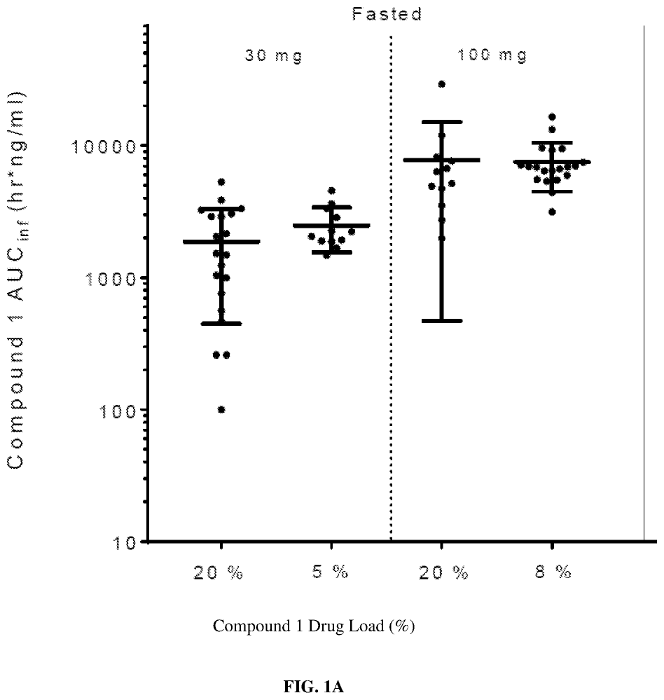

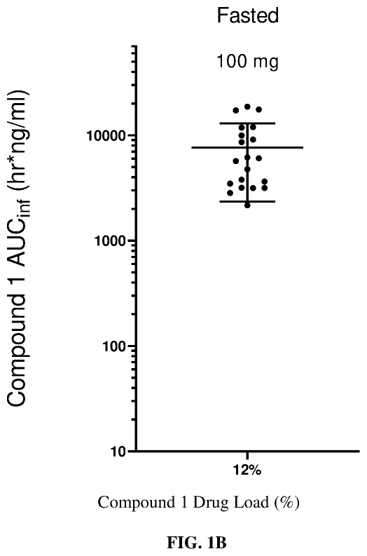

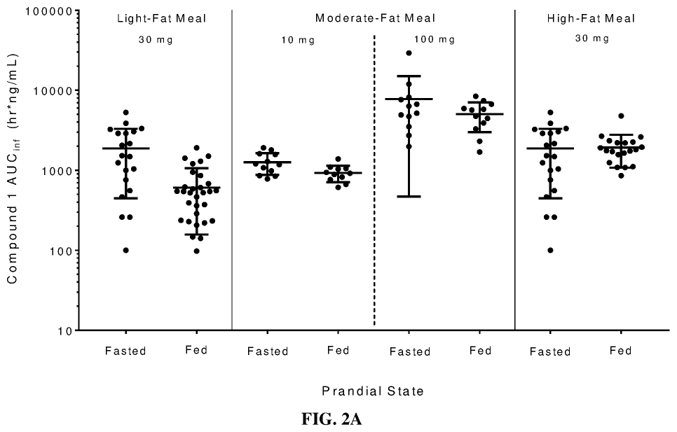

Graphical and statistical summaries of Compound 1 exposure (AUC_{inf}) from the studies listed above are presented in FIG. 1A, FIG. 1B and Table 7 (data presented to 3 significant digits), respectively.

The data shows, for example, that certain drug loads of Compound 1, such as 5% and 8% (or for example, about 5% to about 12% or about 12%) resulted in reduction in variability, and increased Compound 1 exposure compared to that observed with 20% drug load.

TABLE 7
Summary of Compound 1 Exposure and Variability
Across Drug Load of 20%, 5%, and 8%

| Mean (% CV) | | | | | |
|---|---|---|---|---|---|
| Compound 1 AUC_{inf} | | | | | |
| (hr*ng/mL) | | Compound 1 Drug Load | | | |
| Dose | Meal Type | 20% | 5% | 8% | 12% |
| 30 mg | Fasted | 1870 (76.2) | 2470 (37.2) | | |
| 100 mg | Fasted | 7740 (93.9) | | 7480 (40.3) | 7650 (69.4) |

### Example 4: Assessment of the Effect of Meal Type on Compound 1 Exposure and Variability

Single dose pharmacokinetic exposure parameters (AUC_{inf}) of Compound 1 from multiple Phase 1 studies in healthy volunteers which administered Compound 1 under fasted or fed conditions with varying meal types were compared to determine if food and meal type impacted the variability and/or absolute value of Compound 1 systemic exposure. Data used in this analysis are presented in Table 8 below.

TABLE 8
Description of Data Used in this Analysis

| | | | | | Study #, |
|---|---|---|---|---|---|
| Compound 1 | Meal | Compound 1 | N | | Cohort/Treatment |
| Dose (mg) | Type* | Drug Load (%) | (Subjects) | Dosage Form | or Population |
| 30 mg | Fasted | 20% | 20 | Compound 1 | Study B, 1/C |
| | | | | 30 mg Tablet | |
| | Light-Fat | | 30 | Compound 1 | Study B, 3/I |
| | Meal | | | 30 mg Tablet | |
| 10 mg | Fasted | 5% | 12 | Compound 1 | Study A, 1 |
| | | | | 10 mg Tablet | |
| | Moderate-Fat | | 11 | Compound 1 | Study A, 8 |
| | Meal | | | 10 mg Tablet | |
| 100 mg | Fasted | 20% | 12 | Compound 1 | Study A, 3 |
| | | | | 100 mg Tablet | |
| | Moderate-Fat | | 12 | Compound 1 | Study A, 5 |
| | Meal | | | 100 mg Tablet | |
| 30 mg | Fasted | 20% | 20 | Compound 1 | Study B, 1/C |
| | | | | 30 mg Tablet | |
| | High-Fat | | 20 | Compound 1 | Study B, 1/A |
| | Meal | | | 30 mg Tablet + | |
| | | | | ^{†}SEL 18 mg | |
| 100 mg | Fasted | 12% | 20 | Compound 1 | Study D, 10/F |
| | | | | 100 mg Tablet | |
| | Light-Fat | | 20 | Compound 1 | Study D, 10/D |
| | Meal | | | 100 mg Tablet | |
| | High-Fat | | 20 | Compound 1 | Study D, 10/E |
| | Meal | | | 100 mg Tablet | |

*Light-Fat Meal = ~400 kcal with ~20% of calories from fat; Moderate-Fat Meal = ~600 kcal with ~27% of calories from fat; High-Fat Meal = ~800-1000 kcal with ~50% of calories from fat (approximately 150, 250, and 500-600 kcal from protein, carbohydrate, and fat, respectively)
^{†}SEL = Selonsertib. 18 mg of SEL has been previously shown to not alter the PK of Compound 1

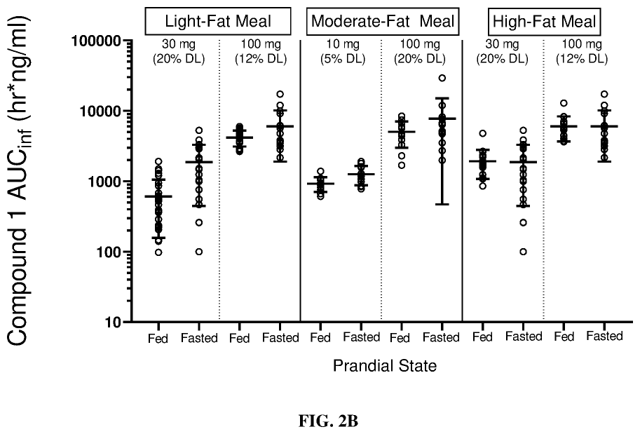

Graphical and statistical summaries of Compound 1 exposure (AUC_{inf}) from the studies listed above are presented in FIG. 2 and Table 9 (data presented to 3 significant digits), respectively. These data show that the effect of food on Compound 1 exposure is meal type dependent with light-fat and moderate-fat meals reducing, but a high-fat meal increasing Compound 1 exposure. Moderate- and high-fat meals reduced the variability of Compound 1 compared to fasted administration irrespective of % drug load, whereas a light-fat meal did not reduce variability in Compound 1 exposure.

TABLE 9
Summary of Compound 1 Exposure, Variability,
and Change in Exposure with Meal-Type

| Compound 1 | Comparison | Compound 1 | | Compound 1 | | | |
|---|---|---|---|---|---|---|---|
| Dose | Fed (N) vs | AUC_{inf} (% CV) | | AUC_{inf} (% CV) | | % GLSM Ratio | |
| (% drug load) | Fasted (N) | Fed | | Fasted | | (90% CI) | |
| 30 mg (20%) | Light-Fat | 608 | (74.2) | 1870 | (76.2) | 37.5 | (24.4, 57.5) |
| | Meal (30) vs | | | | | | |
| | Fasted (20) | | | | | | |
| 10 mg (5%) | Moderate-Fat | 924 | (23.4) | 1260 | (30.4) | 74.9 | (53.9, 104) |
| | meal (11) vs | | | | | | |
| | Fasted (12) | | | | | | |
| 100 mg (20%) | Moderate-Fat | 5020 | (40.3) | 7740 | (93.9) | 76.6 | (55.5, 106) |
| | meal (12) vs | | | | | | |
| | Fasted (12) | | | | | | |
| 30 mg (20%) | High-Fat | 1920 | (44.0) | 1870 | (76.2) | 142 | (97.4, 207) |
| | Meal (20) vs | | | | | | |
| | Fasted (20) | | | | | | |
| 100 mg (12%) | Light-Fat | 4170 | (25.2) | 7650 | (69.4) | 65.7 | (49.9, 86.3) |
| | Meal (20) vs | | | | | | |
| | Fasted (20) | | | | | | |
| 100 mg (12%) | High-Fat | 5930 | (35.7) | 7650 | (69.4) | 91.9 | (81.9, 140) |
| | Meal (20) vs | | | | | | |
| | Fasted (20) | | | | | | |

In Study B, Cohort 1, Compound 1 30 mg was administered with a high fat meal (+selonsertib(SEL)) and in the fasted state to the same subjects in a cross-over manner.

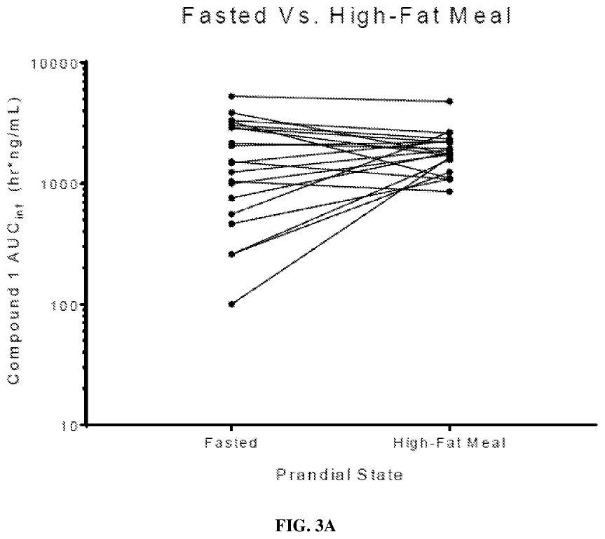

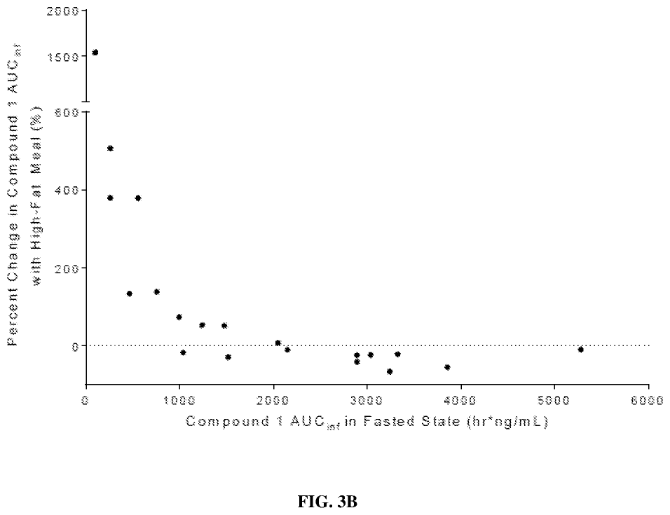

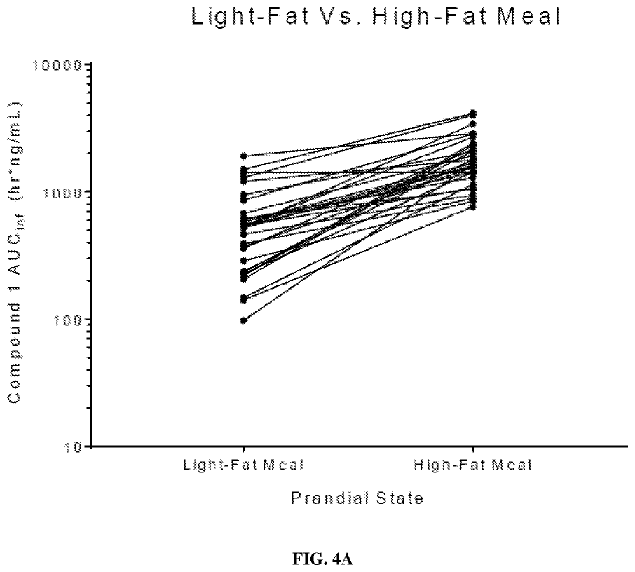

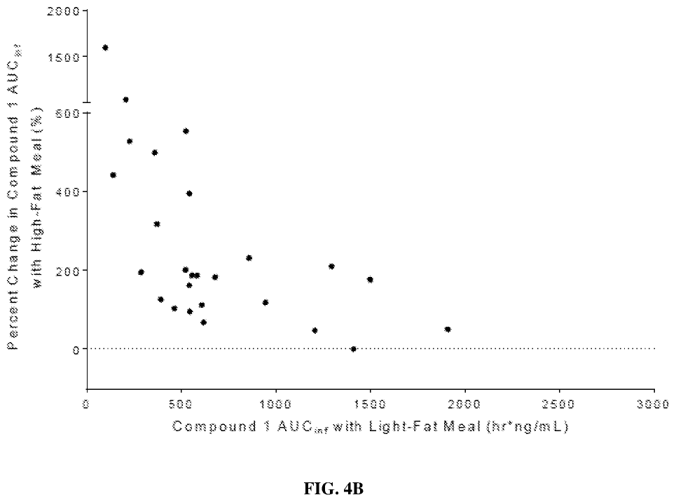

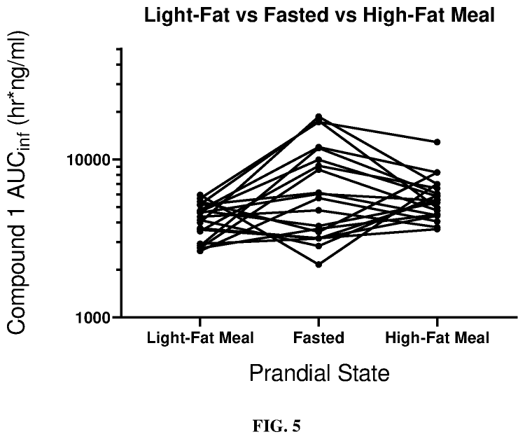

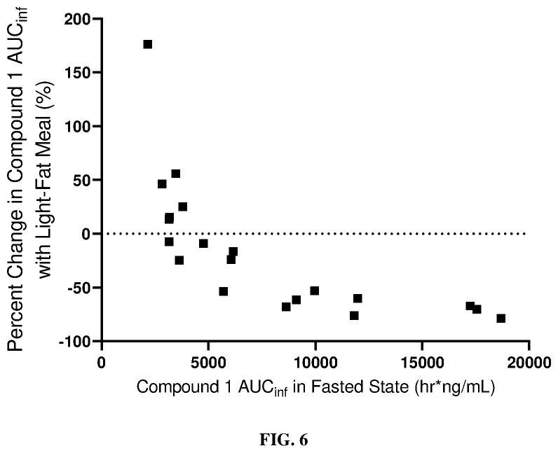

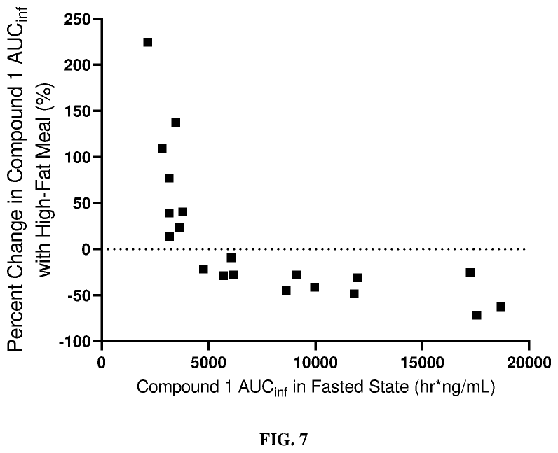

A paired comparison of Compound 1 exposure in these subjects shows that subjects with low exposure when Compound 1 is taken under fasted conditions or with a light-fat meal have greater percent increases in exposure when Compound 1 is taken with a high-fat meal than subjects with high exposure under fasted conditions or with a light-fat meal (FIG. 3A, FIG. 3B, FIG. 4A, FIG. 4B, FIG. 5, FIG. 6, and FIG. 7).

### Example 5: Effect of Acid-reducing Agents in Healthy Subjects

The objective of Cohort 11 was to assess the effect of gastric acid reducing agents (ARAs) on the PK of Compound 1 single agent tablet using famotidine, a representative H2RA. Compound 1 100 mg strength tablets (as free form equivalent) were used. Famotidine was obtained from a commercially available source.

### Dosage and Administration of Study Drug

The study treatments are as follows:

Treatment J: Single dose of Compound 1 (1×100 mg tablet) administered orally in the morning in the fasted state.

Treatment K: Single dose of Compound 1 (1×100 mg tablet) administered orally in the morning 2 hours after famotidine (FAM) (1×40 mg) in the fasted state.

| | |
|---|---|
| Treatment | Study Day |

| Sequence | 1 | 2-8 | 9 |
|---|---|---|---|
| JK | Compound 1 | WO | Compound 1 |
| (N = 10) | 100 mg | | 100 mg + |
| | | | FAM 40 mg |
| KJ | Compound 1 | WO | Compound 1 |
| (N = 10) | 100 mg + | | 100 mg |
| | FAM 40 mg | | |

WO = washout

When study drug administration and intensive PK sampling occurred at the same timepoint, the PK sample were collected at the nominal timepoint and study drug administration occurred after the PK sample collection (within 5 minutes of the nominal timepoint).

### Fasted Administration: Treatments J and K

Study drug(s) were administered in the morning following an overnight fast (no food or drinks except water for at least 10 hours). Subjects continued to fast until after collection of the 4-hour PK sample, relative to the (first) study drug dosing. Additionally, subjects were restricted from water consumption 1 hour before until 2 hours after each study drug dosing, except for the 240 mL given with each study drug administration. Water was optionally consumed by subjects following the 2-hour blood draw for the remainder of the collection period.

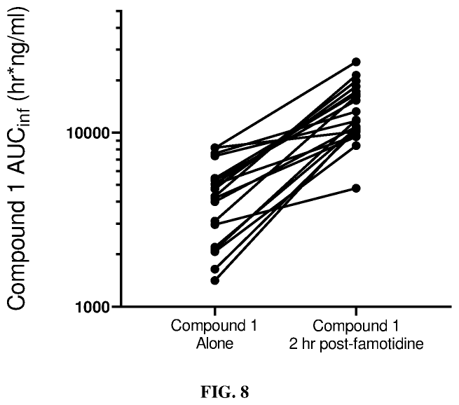

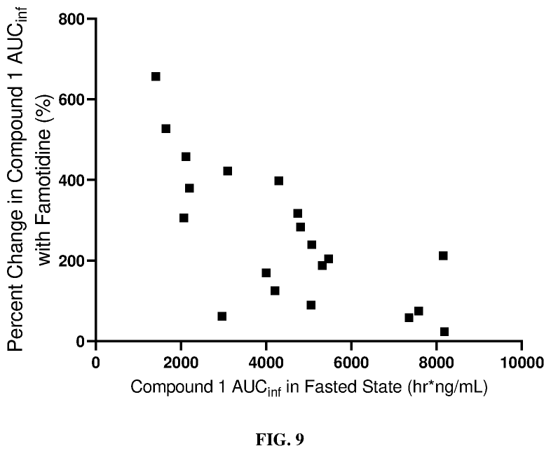

The results of this example show an increase in bioavailability of Compound 1 in famotidine pretreated animals. FIG. 8 shows that there is an increase in bioavailability when Compound 1 is administered two hours after famotidine (a representative histamine 2 receptor antagonist (H2RA)). FIG. 9 shows that there is an increase in exposure (i.e., bioavailability) with famotidine pre-treatment at a 12% drug load of Compound 1. Data is shown in Table 10.

TABLE 10
Compound 1 Exposure, Variability, and Change
in Exposure with Acid Reducing Agent

| | Compound 1 | Compound 1 | |
|---|---|---|---|
| | AUC_{inf} (% CV) | AUC_{inf} (% CV) | |
| Compound 1 | with Famotidine | Alone | |
| Dose | N = 20 | N = 20 | % GLSM Ratio |
| (% drug load) | (Test) | (Reference) | (90% CI) |
| 100 mg (12%) | 13600 (37.3) | 4490 (47.5) | 320 (263, 390) |

It should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1-54. (canceled)

55. A method of treating a condition mediated by nonsteroidal farnesoid X receptor (FXR) in a patient in need thereof comprising administering a tablet comprising from about 5% w/w to about 8% w/w of a Compound 1: 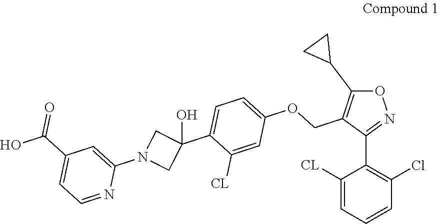
and at least one pharmaceutically acceptable carrier, and wherein the percentage by weight is relative to the total weight of the tablet.

56. The method of claim 55, wherein the tablet comprises about 5% w/w of Compound 1.

57. The method of claim 55, wherein the tablet comprises about 8% w/w of Compound 1.

58. The method of claim 55, wherein the condition mediated by FXR is Non-Alcoholic Steatohepatitis (NASH).

59. The method of claim 58, wherein the tablet comprises about 1 mg to about 200 mg of Compound 1.

60. The method of claim 55, wherein the condition mediated by FXR is primary sclerosing cholangitis (PSC).

61. The method of claim 60, wherein the tablet comprises about 1 mg to about 200 mg of Compound 1.

62. The method of claim 55, wherein the condition mediated by FXR is primary biliary cirrhosis (PBC).

63. The method of claim 55, wherein the tablet is administered with food.

64. The method of claim 55, wherein the tablet is administered with high-fat meal.

65. The method of claim 55, wherein the tablet further comprises a therapeutically effective amount of firsocostat.

66. The method of claim 55, wherein the tablet further comprises from 25% to 60% w/w of microcrystalline cellulose.

67. The method of claim 55, wherein the tablet further comprises from 20% to 60% w/w of lactose monohydrate, mannitol, or a combination thereof.

68. The method of claim 55, wherein the tablet further comprises from 5% to 10% w/w of crospovidone.

69. The method of claim 55, wherein the tablet further comprises from 1% to 2% w/w of magnesium stearate.

70. The method of claim 55, wherein the tablet is a film-coated tablet.
